(19) United States
(12) Patent Application Publication
(10) Pub. No.: US 2026/0176334 A1
(43) Pub. Date: Jun. 25, 2026

(54) SNAP-25 Antibodies and Methods of Using the Same

(71) Applicant: Galderma Holding SA, Zug, (CH)

(72) Inventors: Robert FREDRIKSSON, Uppsala (SE); Lina GUDMUNDS, Örbyhus (SE); Emilia LEKHOLM, Uppsala (SE); Hassani IMANE, Uppsala (SE); Alexandra LENGQUIST, Uppsala (SE); Karin NORDENANKAR, Uppsala (SE); Elena MUSCOLINO, Uppsala (SE); Julia SVENSSON, Uppsala (SE)

(73) Assignee: Galderma Holding SA, Zug, (CH)

(21) Appl. No.: 19/340,173

(22) Filed: Sep. 25, 2025

Related U.S. Application Data
(60) Provisional application No. 63/699,563, filed on Sep. 26, 2024.

(51) Int. Cl. C07K 16/00; G01N 33/569

(52) U.S. Cl. CPC C07K 16/00; G01N 33/56911; C07K 2317/565

(57) ABSTRACT

Antibodies that bind to SNAP-25 are provided. Botulinum toxin potency screening assays that utilize anti-SNAP-25 antibodies are also provided.

### CROSS-REFERENCE TO RELATED APPLICATION

This application claims priority to U.S. Patent Application No. 63/699,563, filed Sep. 26, 2024, the entire contents of which are hereby incorporated by reference herein.

### SEQUENCE LISTING

The instant application contains a Sequence Listing which has been submitted electronically in XML format and is hereby incorporated by reference in its entirety. Said XML copy, created on Jan. 29, 2026, is named 105153-1427_SL.xml and is 16,261 bytes in size.

### FIELD OF THE INVENTION

The present disclosure relates to the field of botulinum toxin potency screening for preparing compositions comprising botulinum toxins.

### BACKGROUND

The following discussion is provided to aid the reader in understanding the disclosure and is not admitted to describe or constitute prior art thereto.

The botulinum neurotoxins (BoNTs) are a family of structurally similar protein neurotoxins which act on the peripheral nervous system to block neuromuscular transmission. These neurotoxins are extremely potent, and with a human lethal dose on the order of sub-micrograms. Assays for the botulinum neurotoxins are currently used in both the food and pharmaceutical industry. The food industry employs assays for the botulinum neurotoxins to validate new food packaging methods and to ensure food safety. With the growing clinical use of the botulinum toxins, the pharmaceutical industry requires accurate assays for these toxins for both product formulation and quality control.

The current standard assay for assessing BoNT activity is the mouse lethality test. This test has been the industry standard for many years, though over the past 10 years a number of immunoassay methods have been developed in an attempt to replace the mouse test in the majority of applications. Nevertheless, reliable and reproducable alternatives to traditional mouse assays are still needed.

### SUMMARY OF THE DISCLOSURE

The present disclosure provides antibodies and antigen binding fragments thereof that bind to Synaptosomal-Associated Protein, 25 kDa (SNAP-25) and cell-based methods of determining the potency of botulinum neurotoxins (BoNTs) without having to rely on LD₅₀ experimentation on mice.

In one aspect, the present disclosure provides antibody fragment that binds to Synaptosomal-Associated Protein, 25 kDa (SNAP-25) and comprises a heavy chain region and optionally comprises a light chain region, the heavy chain region comprising a complementarity determining region 1 (CDRH1) comprising the amino acid sequence of SYAMH (SEQ ID NO: 1), a CDRH2 comprising the amino acid sequence of VISYDGSYTYYADSVKG (SEQ ID NO: 2), and a CDRH3 comprising the amino acid sequence of ISPQARGADFDL (SEQ ID NO: 3), and the light chain region comprising a CDRL1 comprising the amino acid sequence of SGDAIPSKYVS (SEQ ID NO: 4), a CDRL2 comprising the amino acid sequence of RDSDRPS (SEQ ID NO: 5), and a CDRL3 comprising the amino acid sequence of GSWDMHLWV (SEQ ID NO: 6).

In some embodiments, the antibody fragment is selected from a Fd, a Fv, a Fab, a Fab′, a F(ab′)₂, and a scFv.

In some embodiments, the heavy chain region comprises a portion of an amino acid sequence that comprises at least 90%, 91%, 92%, 93%, 94%, 95%, 96%, 97%, 98%, 99%, or 100% sequence identity to

(SEQ ID NO: 7)
EVQLLESGGGLVQPGGSLRLSCAASGFTFSSYAMHWVRQAPGKGLEWVSV
ISYDGSYTYYADSVKGRFTISRDNSKNTLYLQMNSLRAEDTAVYYCARIS
PQARGADFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKS
or
(SEQ ID NO: 8)
EVQLLESGGGLVQPGGSLRLSCAASGFTFSSYAMHWVRQAPGKGLEWVSV
ISYDGSYTYYADSVKGRFTISRDNSKNTLYLQMNSLRAEDTAVYYCARIS
PQARGADFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSEFDYKDDDDKGGSVPTIVMVDAYKRYK
GAPHHHHHH.

In some embodiments, the light chain region comprises a portio of an amino acid sequence that comprises at least 90%, 91%, 92%, 93%, 94%, 95%, 96%, 97%, 98%, 99%, or 100% sequence identity to

(SEQ ID NO: 9)
DIELTQPPSVSVSPGQTASITCSGDAIPSKYVSWYQQKPGQAPVLVIYRD
SDRPSGIPERFSGSNSGNTATLTISGTQAEDEADYYCGSWDMHLWVFGGG
TKLTVLGQPKAAPSVTLFPPSSEELQANKATLVCLISNFYPGAVTVAWKA
DSSPVKAGVETTTPSKQSNNKYAASSYLSLTPEQWKSHRSYSCQVTHEGS
TVEKTVAPTEA.

In some embodiments, the heavy chain region comprises a portion of an amino acid sequence with 1-10 amino acid substitutions, deletions, or additions to the amino acid sequence of SEQ ID NO: 7. In some embodiments, the light chain region comprises a portion of an amino acid sequence with 1-10 amino acid substitutions, deletions, or additions to the amino acid sequence of SEQ ID NO: 9.

In some embodiments, the heavy chain region comprises a portion of the amino acid sequence of SEQ ID NO: 7. In some embodiments, the light chain region comprises a portion of the amino acid sequence of SEQ ID NO: 9.

In some embodiments, the antibody fragment is a fragment of a monoclonal antibody. In some embodiments, the antibody fragment is a fragment of an IgG antibody.

In some embodiments, the antibody fragment is conjugated to a detectable label. In some embodiments, the detectable label comprises a radioisotope, a dye, a fluorophore, a protein, or an enzyme.

In some embodiments, the antibody fragment is encoded by a nucleic acid. In some embodiments, the nucleic acid encoding the antibody fragment's heavy chain region sequence comprises at least 90%, 91%, 92%, 93%, 94%, 95%, 96%, 97%, 98%, 99%, or 100% sequence identity to SEQ ID NO: 10, and the nucleic acid encoding the antibody fragment's light chain region sequence comprises at least 90%, 91%, 92%, 93%, 94%, 95%, 96%, 97%, 98%, 99%, or 100% sequence identity to SEQ ID NO: 11.

In some embodiments, the nucleic acid encoding a portion of the antibody fragment's heavy chain region sequence comprises SEQ ID NO: 10, and the nucleic acid encoding a portion of the antibody fragment's light chain region sequence comprises SEQ ID NO: 11.

In some embodiments, the nucleic acid comprises SEQ ID NO: 10 and 11. In some embodiments, the present disclosure also provides an expression vector operably linked to a promoter comprises SEQ ID NO: 10, SEQ ID NO: 11, or SEQ ID NO: 10 and 11. In such embodiment, the promoter preferably drives expression of sequences comprising SEQ ID NO: 10, SEQ ID NO: 11, or SEQ ID NO: 10 and 11.

In another aspect, the disclosure is broadly drawn to a method of determining potency of BoNTs, the method comprising: (a) incubating in a first series of containers a first population of cells that express SNAP-25 with a serial dilution of a first BoNT sample of known potency and incubating in a second series of containers a second population of cells that express SNAP-25 with a serial dilution of a second BoNT sample of unknown potency, wherein each incubation is for a defined period of time, (b) after the incubation, separating cleaved SNAP-25 from uncleaved SNAP-25 from each of the first population of cells and the second population of cells, and (c) contacting cleaved and uncleaved SNAP-25 from each of the first population of cells and the population of cells with an anti-Synaptosomal-Associated Protein, 25 kDa (SNAP-25) antibody or antigen-binding fragment thereof that comprises a heavy chain region and optionally comprises a light chain region, the heavy chain region comprising a complementarity determining region 1 (CDRH1) comprising the amino acid sequence of SYAMH (SEQ ID NO: 1), a CDRH2 comprising the amino acid sequence of VISYDGSYTYYADSVKG (SEQ ID NO: 2), and a CDRH3 comprising the amino acid sequence of ISPQARGADEDL (SEQ ID NO: 3), and the light chain region comprising a CDRL1 comprising the amino acid sequence of SGDAIPSKYVS (SEQ ID NO: 4), a CDRL2 comprising the amino acid sequence of RDSDRPS (SEQ ID NO: 5), and a CDRL3 comprising the amino acid sequence of GSWDMHLWV (SEQ ID NO: 6), and quantifying the relative amount of cleaved and uncleaved SNAP-25 in each of the first population of cells and the second population of cells. In some embodiments, a third BoNT sample, a quality control sample, of known potency, is distributed to a third series of containers in a serial dilution and utilized as a positive control.

In some embodiments, separating cleaved SNAP-25 from uncleaved SNAP-25 from each of the first population of cells and the second population of cells comprises gel electrophoresis. In some embodiments, quantifying the relative amount of cleaved and uncleaved SNAP-25 in each of the first population of cells and the second population of cells comprises performing Western blotting and densitometric analysis.

In some embodiments, the period of time is for at least 6, 12, 16, 20, 24, 32, 40, 48, or 56 hours. In some embodiments, the at least two containers each comprise a plurality of wells. In some embodiments, the at least two different BoNT samples are serially diluted across the plurality of wells. In some embodiments, the at least two containers are tissue culture plates. In some embodiments, the at least two containers are 48-, 96-, 384-, or 1536-well plates.

In some embodiments, the cells are adhered or attached to the at least two containers. In some embodiments, the cells natively express SNAP-25. In some embodiments, the cells express a heterologous SNAP-25. In some embodiments, the cells are non-neuronal cells. In some embodiments, the cells are genetically modified. In some embodiments, the cells are neuronal cells. In some embodiments, the neuronal cells are motor neurons.

In some embodiments, the cells are treated with a non-proliferation agent. In some embodiments, the non-proliferation agent inhibits γ-secretase. In some embodiments, the non-proliferation agent is DAPT. In some embodiments, a protease inhibitor is added to the at least two containers upon conclusion of (a) to prevent degradation of SNAP-25.

In some embodiments, the cells are lysed after incubating the cells with the BoNT. In some embodiments, the cells are lysed by sonication. In some embodiments, the cells are lysed by addition of a lysis agent. In some embodiments, the lysis agent comprises a detergent. In some embodiments, the BoNT samples are selected from BoNT/A, BoNT/E, and BoNT/C.

The following detailed description is exemplary and explanatory, and is intended to provide further explanation of the invention.

### BRIEF DESCRIPTION OF THE FIGURES

FIG. 1 depicts an image obtained through Western blotting using 0.8 μg/mL of the presently disclosed antibody. In the left lane, purified SNAP-25 is detected. In the right lane, uncleaved (upper band) and cleaved (lower band) native SNAP-25 is detected.

FIG. 2A-2B depict images obtained through Western blotting using 0.8 μg/mL (FIG. 2A) and 0.4 μg/mL (FIG. 2A) of the presently disclosed antibody. Native SNAP-25 expressed in motor neurons is detected at both concentrations.

FIG. 3 depicts an image obtained through Western blotting using 0.8 μg/mL of antibody directly labeled with horse radish peroxidase enzyme (HRP). In the left lane, purified SNAP-25 is detected; in the right lane, uncleaved (upper band) and cleaved (lower band) native SNAP-25 from motor neurons is detected.

FIG. 4A-4C depict graphs showing the sensitivity of candidate antibodies AbD56219sco-1, AbD56220sco-1, AbD56221sco-1, AbD56222sco-1, AbD56223sco-1 (FIG. 4A), AbD56224ad-1, AbD56225ad-1, AbD56226ad-1, AbD56227ad-1, AbD56228ad-1, AbD56229ad-1, AbD56230ad-1 (FIG. 4B), AbD56359ad-1, AbD56360ad-1, AbD56361ad-1, and AbD56424ad-1 (FIG. 4C) in a direct ELISA assay.

FIG. 5A-5O depict images obtained through Western blotting using candidate antibodies AbD56219sco-1 (FIG. 5A), AbD56220sco-1 (FIG. 5B), AbD56221sco-1 (FIG. 5C), AbD56222sco-1 (FIG. 5D), AbD56223sco-1 (FIG. 5E), AbD56224ad-1 (FIG. 5F), AbD56225ad-1 (FIG. 5G), AbD56226ad-1 (FIG. 5H), AbD56227ad-1 (FIG. 5I), AbD56228ad-1 (FIG. 5J), AbD56229ad-1 (FIG. 5K), AbD56230ad-1 (FIG. 5L), AbD56360ad-1 (FIG. 5M), AbD56424ad-1 (FIG. 5N), and AbD56359ad-1 (FIG. 5O). In the left lanes, purified SNAP-25 is detected; in the right lanes, native SNAP-25 from motor neuron cultures is detected; in the far-right lanes (when present), the native SNAP-25 lane was removed from the blot and re-imaged with a longer exposure time for 5B-5O but not 5A.

FIG. 6A-6B depict graphs showing an unconstrained (FIG. 6A) and constrained (FIG. 6B) model fit of cell-based assay dose response data. Data was obtained by Western blotting using the presently disclosed antibody.

FIG. 7A-7D depict images obtained through Western blotting showing uncleaved (upper band) and cleaved (lower band) native SNAP-25 detected with the presently disclosed antibody following a cell-based assay on a reference sample (left Western blots; 109 U/mL BoNT/A prior to serial dilution), a cell based assay with reduced potency BoNT/A (right Western blots; 100 U/mL (FIG. 7A), 80 U/mL (FIG. 7B), 60 U/mL (FIG. 7C), 40 U/mL (FIG. 7D) BoNT/A prior to serial dilution), and graphs showing the unconstrained and constrained model fits of cell-based assay dose response data.

FIG. 8 depicts a graph showing the linear regression of FIG. 7A-7D cell-based assay relative potency results versus expected results.

### DETAILED DESCRIPTION

Alternatives to assessing potency of botulinum toxins that do not rely on animal models, such as the conventional mouse LD₅₀ assessment, are needed. One such assay operates by addition of a test sample to a plate or column to which is attached an antibody that binds to toxin present in the sample. A further antibody is typically used to detect bound toxin. These enzyme-linked immunoassays (ELISAs) have the advantages that they are specific to one botulinum toxin type and can be performed rapidly, in less than 2 hours. The ELISAs, however, suffer from several drawbacks: (1) they do not measure the biological activity of the toxins, (2) they cannot distinguish between active and inactive toxin, and (3) due to antigenic variations, some toxins are not detected by these assays which therefore give rise to false negatives. The present disclosure provides a different cell-based assessment that is both accurate and reproducable, as well as antibodies that can be used in the disclosed cell-based assay.

Specifically, antibodies that bind Synaptosomal-Associated Protein, 25 kDa (SNAP-25) are provided. Antibody heavy chains and light chains that are capable of forming antibodies that bind SNAP-25 are also provided. In addition, antibodies, heavy chains, and light chains comprising one or more particular complementarity determining regions (CDRs) are provided. Polynucleotides encoding antibodies that bind to SNAP-25 are provided. Polynucleotides encoding antibody heavy chains or lights chains are also provided. Methods of producing and/or purifying antibodies to SNAP-25 are provided. Assays using the antibodies are provided. Such methods include, but are not limited to, method of quantifying SNAP-25 cleavage by botulinum neurotoxins.

### Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

As used herein, a “control” is an alternative sample used in an experiment for comparison purpose. A control can be “positive” or “negative.” A “control sample” or “reference sample” as used herein, refers to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, “SNAP25” and “SNAP-25” refer to a peptide sequence, or fragment thereof, of a human SNAP25 protein (Entrez 6616; UniProt P60880).

As used herein, “VAMP-1” and “VAMP1” refer to a peptide sequence, or fragment thereof, of a human VAMP1 protein (Entrez 6843; UniProt P23763).

As used herein, “VAMP-2” and “VAMP2” refer to a peptide sequence, or fragment thereof, of a human VAMP2 protein (Entrez 6844; UniProt P63027).

As used herein, “VAMP-3” and “VAMP3” refer to a peptide sequence, or fragment thereof, of a human VAMP3 protein (Entrez 9341; UniProt Q15836).

As used herein, “syntaxin” and “syntaxin 1a” refers to a peptide sequence, or fragment thereof, of a human syntaxin protein (Entrez 6804; UniProt Q16623).

As used herein, “botulinum toxin,” “botulinum neurotoxin,” and “BoNT” are used interchangeably to refer to any of the neurotoxic proteins produced by the bacterium Chlostridium botulinum. The neurotoxic proteins include botulinum neurotoxin A, B, C, D, E, F, G, H, BoNT/En, BoNT/X, PMP1, BtNT, Cp1 and BoNT/Wo.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a SNAP-25 epitope is an antibody that binds this epitope with greater affinity, avidity, more readily, and/or with greater duration than it binds to other SNAP-25 epitopes or non-SNAP-25 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term “CDR” denotes a complementarity determining region as defined by at least one manner of identification to one of skill in the art.

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an δ constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgAQ1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCDR1, framework (FR) 2, LCDR2, FR3, and LCDR3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Non-limiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

A “chimeric antibody” as used herein refers to an antibody in which a portion of the heavy and/or light chain is derived from a particular source or species, while at least a part of the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species. The chimeric construct can also be a functional fragment, as noted above.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

An “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

An amino acid substitution may include but are not limited to the replacement of one amino acid in a polypeptide with another amino acid. Exemplary conservative substitutions are shown below. Amino acid substitutions may be introduced into an antibody of interest and the products screened for a desired activity, for example, retained/improved antigen binding, decreased immunogenicity, or improved ADCC or CDC.

| Original | Exemplary |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively.

### Botulinum Toxins (BoNTS)

BoNTs have been traditionally classified into seven serotypes distinguishable with animal antisera and designated with the letters, A, B, C, D, E, F, G, and H. Molecular genetic analysis has led to the discovery of genes encoding for many novel BoNTs, include subtypes within each of the serotypes, expanding the known genus of BoNTs drastically over the last decade. While the first discovered BoNTs were known to be produced by Clostridium botulinum, multiple Clostridium species produce BoNTs. In some aspects, BoNTs are produced by C. botulinum, C. baratii, C. butyricum, and C. argentinense. In some aspects, BoNT serotypes are selected from A, B, C, D, E, F, G, H. In some aspects, BoNTs enzymatically cleave SNAP-25, VAMP1, VAMP2, VAMP3, syntaxin. In some aspects, SNAP-25 is cleaved by BoNT/A, BoNT/E, and BoNT/C. In some aspects, VAMP1 is cleaved by BoNT/B, BoNT/F, BoNT/D, BoNT/G, and BoNT/H. In some aspects, VAMP2 is cleaved by BoNT/B, BoNT/F, BoNT/D, BoNT/G, and BoNT/H. In some aspects, VAMP3 is cleaved by BoNT/B, BoNT/F, BoNT/D, BoNT/G, and BoNT/H. In some aspects, syntaxin is cleaved by BoNT/C.

In some aspects, the BoNTs are chimeric. In some aspects, chimeric BoNTs are selected from BoNT/DC, BoNT/CD, and BoNT/FA. In some aspects, BonT/A comprises subtypes selected from A1, A2, A3, A4, A5, A6, A7, and A8. In some aspects, BoNT/B comprises subtypes selected from B1, B2, B3, B4, B5, B6, B7, and B8. In some aspects, BoNT/E comprises subtypes selected from E1, E2, E3, E4, E5, E6, E7, E8, E9, E10, E11, and E12. In some aspects, BoNT/F comprises subtypes selected from F1, F2, F3, F4, F5, F6, F7, and F8. In some aspects, BoNT/G comprises subtype G. In some aspects, BoNT/H comprises subtypes selected from H, F/A, and H/A.

In some aspects, the BoNTs are multivalent, such as bivalent and trivalent. In some aspects, multivalent BoNTs comprise BoNT/Ba, BoNT/Bf, BoNT/Ab, BoNT/Af, BoNT/A (B), and BoNT/A2F4F5.

### Anti-SNAP-25 Antibodies

The present disclosure provides antibodies that bind to SNAP-25 (i.e., “anti-SNAP-25 antibodies”). The disclosed antibodies can bind selectively to SNAP-25 and be used to determine the potency of BoNTs.

Anti-SNAP-25 antibodies described herein can be obtained by any means, including in vitro sources (e.g., a hybridoma or a cell line producing an antibody recombinantly) and in vivo sources (e.g., rodents, rabbits, humans, etc.). The disclosed antibodies may be human, humanized (partially or fully), or chimeric. Human, partially humanized, fully humanized, and chimeric antibodies can be made by methods known in the art, such as using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9): 1117-25 (2005), and Lonberg, Handb. Exp. Pharmacol., 181:69-97 (2008))

Anti-SNAP-25 antibodies disclosed herein generally will be monoclonal, recombinant, or both. Monoclonal antibodies (mAbs) may be obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with a SNAP-25 antigen. Recombinant antibodies may be obtained by methods known in the art, for example, using phage display technologies, yeast surface display technologies (Chao et al., Nat. Protoc., 1(2): 755-68 (2006)), mammalian cell surface display technologies (Beerli et al., PNAS, 105(38): 14336-41 (2008), and/or expressing or co-expressing antibody polypeptides. Other techniques for making antibodies are known in the art, and can be used to obtain antibodies used in the methods described herein.

Typically, an antibody consists of four polypeptides: two identical copies of a heavy (H) chain polypeptide and two copies of a light (L) chain polypeptide. Typically, each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody, and comprise complementarity determining regions (CDR).

The term “antibody fragment,” as used herein, refer to one or more portions of a SNAP-25-binding antibody that exhibits the ability to bind SNAP-25. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H}CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples include binding domain immunoglobulin fusion proteins comprising (i) a binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization.

The disclosed antibodies may belong to a class of antibody selected from IgG, IgM, IgA, IgE, and IgD. More specifically, the disclosed antibodies may be an IgG1, IgG2, IgG3, or IgG4. In some embodiments, the disclosed antibodies may comprise all or part of the constant regions, framework regions, or a combination thereof of an IgG, IgM, IgA, IgE, or IgD antibody. For instance, a disclosed antibody may comprise an IgG1 immunoglobulin structure that can be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). In some embodiments, the anti-SNAP-25 antibody may be mammalian, human, humanized, or chimeric.

Variable heavy and variable light chain amino acid and nucleic acid sequences of an exemplary anti-SNAP-25 antibody are disclosed in Table 1 and Table 2, respectively.

TABLE 1
Amino acid sequences.

| CDR or | | SEQ |
|---|---|---|
| Chain | Amino Acid | ID |
| Region | Sequence | NO: |
| CDRH1 | SYAMH | 1 |
| CDRH2 | VISYDGSYTYYADSVKG | 2 |
| CDRH3 | ISPQARGADFDL | 3 |
| CDRL1 | SGDAIPSKYVS | 4 |
| CDRL2 | RDSDRPS | 5 |
| CDRL3 | GSWDMHLWV | 6 |
| Heavy | EVQLLESGGGLVQPGGS | 7 |
| chain | LRLSCAASGFTFS | |
| region | SYAMH | |
| | WVRQAPGKGLEWVS | |
| | VISYDGSYTYYADSVKG | |
| | RFTISRDNSKNTLYLQM | |
| | NSLRAEDTAVYYCAR | |
| | ISPQARGADFDL | |
| | WGQGTLVTVSSASTKGP | |
| | SVFPLAPSSKSTSGGTA | |
| | ALGCLVKDYFPEPVTVS | |
| | WNSGALTSGVHTFPAVL | |
| | QSSGLYSLSSVVTVPSS | |
| | SLGTQTYICNVNHKPSN | |
| | TKVDKKVEPKS | |
| Heavy | EVQLLESGGGLVQPGGS | 8 |
| chain | LRLSCAASGFTFS | |
| region | SYAMHW | |
| (with | VRQAPGKGLEWVS | |
| tags) | VISYDGSYTYYADSVKG | |
| | RFTISRDNSKNTLYLQM | |
| | NSLRAEDTAVYYCAR | |
| | ISPQARGADFDL | |
| | WGQGTLVTVSSASTKGP | |
| | SVFPLAPSSKSTSGGTA | |
| | ALGCLVKDYFPEPVTVS | |
| | WNSGALTSGVHTFPAVL | |
| | QSSGLYSLSSVVTVPSS | |
| | SLGTQTYICNVNHKPSN | |
| | TKVDKKVEPKSEF | |
| | DYKDDDDKGGS | |
| | VPTIVMVDAYKRYK | |
| | GAPHHHHHH | |
| Light | DIELTQPPSVSVSPGQT | 9 |
| chain | ASITC | |
| region | SGDAIPSKYVS | |
| | WYQQKPGQAPVLVIY | |
| | RDSDRPS | |
| | GIPERFSGSNSGNTATL | |
| | TISGTQAEDEADYYC | |
| | GSWDMHLWV | |
| | FGGGTKLTVLGQPKAAP | |
| | SVTLFPPSSEELQANKA | |
| | TLVCLISNFYPGAVTVA | |
| | WKADSSPVKAGVETTTP | |
| | SKQSNNKYAASSYLSLT | |
| | PEQWKSHRSYSCQVTHE | |
| | GSTVEKTVAPTEA | |

Complementarity determining regions (CDRs) are shown in bold, underlined text. CDR annotation was made according to Kabat numbering.
Tags are shown in bold, italicized text.

TABLE 2
Nucleic acid sequences.

| | | SEQ |
|---|---|---|
| Chain | | ID |
| Region | Nucleic Acid Sequence | NO: |
| Heavy | GAAGTGCAATTGCTGGAAAGCGGCGGTGGCCTGGT | 10 |
| chain | GCAGCCGGGTGGCAGCCTGCGTCTGAGCTGCGCGG | |
| region | CGTCCGGATTCACCTTTTCTTCTTACGCTATGCATT | |
| (with | GGGTGCGCCAGGCCCCGGGCAAAGGTCTCGAGTGG | |
| tags) | GTTTCCGTTATCTCTTACGACGGTTCTTACACCTAC | |
| | TATGCGGATAGCGTGAAAGGCCGCTTTACCATCAG | |
| | CCGCGATAATTCGAAAAACACCCTGTATCTGCAAA | |
| | TGAACAGCCTGCGTGCGGAAGATACGGCCGTGTAT | |
| | TATTGCGCGCGTATCTCTCCGCAGGCTCGTGGTGCT | |
| | GACTTCGATCTGTGGGGCCAAGGCACCCTGGTGAC | |
| | TGTTAGCTCAGCGTCGACCAAAGGCCCGAGCGTGT | |
| | TTCCGCTGGCCCCGAGCAGCAAAAGCACCAGCGGC | |
| | GGCACCGCCGCACTGGGCTGCCTGGTGAAAGATTA | |
| | TTTCCCGGAACCAGTGACCGTGAGCTGGAACAGCG | |
| | GTGCCCTGACCAGCGGCGTGCATACCTTTCCGGCG | |
| | GTGCTGCAAAGCAGCGGCCTGTATAGCCTGAGCAG | |
| | CGTTGTGACCGTGCCGAGCAGCAGCCTGGGCACCC | |
| | AGACCTATATTTGCAACGTCAACCATAAACCGAGC | |
| | AACACCAAAGTCGATAAAAAAGTCGAACCGAAAA | |
| | GCGAATTCGACTATAAAGATGACGATGACAAAGGT | |
| | GGTTCTGTTCCTACTATTGTTATGGTGGACGCCTAC | |
| | AAACGCTATAAGGGCGCGCCGCACCATCATCACCA | |
| | TCAC | |
| Light | GATATCGAACTGACCCAGCCGCCGAGCGTGAGCGT | 11 |
| chain | GAGCCCGGGCCAGACCGCGAGCATTACCTGTAGCG | |
| region | GCGATGCTATCCCGTCTAAATACGTTTCTTGGTACC | |
| | AGCAGAAACCGGGCCAGGCGCCGGTGCTGGTGATC | |
| | TACCGTGACTCTGACCGTCCAAGCGGCATCCCGGA | |
| | ACGTTTTAGCGGATCCAACAGCGGCAACACCGCGA | |
| | CCCTGACCATTAGCGGCACCCAGGCGGAAGACGAA | |
| | GCGGATTATTACTGCGGTTCTTGGGACATGCATCTG | |
| | TGGGTGTTTGGCGGCGGCACGAAGTTAACCGTTCT | |
| | TGGCCAGCCGAAAGCCGCCCCAAGCGTGACCCTGT | |
| | TTCCGCCGAGCAGCGAAGAACTGCAAGCCAACAA | |
| | AGCCACCCTGGTTTGCCTGATCAGCAATTTTTATCC | |
| | GGGTGCCGTGACCGTGGCCTGGAAAGCCGATAGCA | |
| | GCCCGGTGAAAGCCGGCGTGGAAACCACCACCCCG | |
| | AGCAAACAGAGCAACAACAAATATGCCGCCAGCA | |
| | GCTATCTGAGCCTGACCCCGGAACAGTGGAAAAGC | |
| | CATCGCAGCTATAGTTGTCAAGTGACCCATGAAGG | |
| | CAGCACCGTGGAAAAAACCGTGGCCCCGACCGAG | |
| | GCC | |

The present disclosure provides anti-SNAP-25 antibodies comprising the same CDR sequences and/or the same variable region sequences as one or more of the sequences disclosed in Table 1. For example, in some embodiments, the disclosed anti-SNAP-25 antibody may comprise a heavy chain variable region comprising the CDRs of SEQ ID NO: 1-3 and a light chain variable region comprising the CDRs of SEQ ID NO: 4-6. In some embodiments, the disclosed anti-SNAP-25 antibody may comprise a heavy chain variable region of SEQ ID NO: 7 and a light chain variable region of SEQ ID NO: 9. In some embodiments, the disclosed anti-SNAP-25 antibody may comprise a heavy chain variable region of SEQ ID NO: 8 and a light chain variable region of SEQ ID NO: 9.

In some embodiments, the anti-SNAP-25 antibody may comprise a heavy chain comprising a CDRH1 comprising SYAMH (SEQ ID NO: 1), a CDRH2 comprising VISYDGSYTYYADSVKG (SEQ ID NO: 2), and a CDRH3 comprising ISPQARGADFDL (SEQ ID NO: 3); and a light chain comprising a CDRL1 comprising SGDAIPSKYVS (SEQ ID NO: 4), a CDRL2 comprising RDSDRPS (SEQ ID NO: 5), and a CDRL2 comprising GSWDMHLWV (SEQ ID NO: 6). In some embodiments, the heavy chain comprises a tag, such as an HA tag, a myc tag, a FLAG tag, a poly-histidine tag, or any combination thereof. In some embodiments, the tag may comprise the amino acid sequence of DYKDDDDK, VPTIVMVDAYKRYK, or HHHHHH. In some embodiments, the heavy chain comprises multiple tags. In some embodiments, the anti-SNAP-25 antibody is AbD56219sco-1.

In some embodiments, an anti-SNAP-25 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7 or 8. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-SNAP-25 antibody comprising that sequence retains the ability to bind to SNAP-25. In some embodiments, a total of 1 to 10 amino acids (e.g., 1, 2, 3, 4, 5, 6, 7, 8, 9, or 10 amino acids) have been substituted, inserted and/or deleted in SEQ ID NO: 2, 4, 6, 10, or 12. In some embodiments, substitutions, insertions, or deletions occur in regions outside the CDRs (that is, in the FRs).

In some embodiments, an anti-SNAP-25 antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 9. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-SNAP-25 antibody comprising that sequence retains the ability to bind to SNAP-25. In some embodiments, a total of 1 to 10 amino acids (e.g., 1, 2, 3, 4, 5, 6, 7, 8, 9, or 10 amino acids) have been substituted, inserted and/or deleted in SEQ ID NO: 9. In some embodiments, substitutions, insertions, or deletions occur in regions outside the CDRs (that is, in the FRs).

The present disclosure further provides anti-SNAP-25 antibodies encoded by nucleic acid sequences disclosed in Table 2. In some embodiments, the nucleic acid encoding the antibody or antigen binding fragment's heavy chain region sequence comprises at least 90%, 91%, 92%, 93%, 94%, 95%, 96%, 97%, 98%, 99%, or 100% sequence identity to SEQ ID NO: 10, and the nucleic acid encoding the antibody or antigen binding fragment's light chain region sequence comprises at least 90%, 91%, 92%, 93%, 94%, 95%, 96%, 97%, 98%, 99%, or 100% sequence identity to SEQ ID NO: 11. In some embodiments, the nucleic acid comprises both SEQ ID NO: 10 and 11. In some embodiments, an expression vector comprises the nucleic acid operably linked to a promoter. In such embodiment, the promoter preferably drives expression of sequences comprising at least 90%, 91%, 92%, 93%, 94%, 95%, 96%, 97%, 98%, 99%, or 100% sequence identity to SEQ ID NO: 10, SEQ ID NO: 11, or SEQ ID NO: 10 and 11.

As noted above, the disclosed antibodies can bind to various SNAP-25 proteins, including purified human SNAP-25, recombinant SNAP-25, and SNAP-25 cleaved by a BoNT.

### Determining Relative Potency of Botulinum Toxins

Botulinum neurotoxins are known to possess highly specific zinc-endopeptidase activities within their light sub-units. Depending on the neurotoxin type, these act to cleave small proteins within the nerve cell which are involved in neurotransmitter release. Botulinum types A (BoNT/A), E (BoNT/E), and C (BoNT/C) toxins cleave the protein, SNAP-25. Botulinum types B, D, F and G and tetanus toxins cleave vesicle-associated membrane protein (VAMP—also called synaptobrevin). Botulinum type C toxin cleaves the protein syntaxin.

In the development of further toxin assays, various procedures have been devised for the evaluation of endopeptidase activities. Liquid chromatography procedures are known and are based on resolution of the peptide product and subsequent evaluation. These procedures are time-consuming, expensive, and do not lend themselves readily to automation. It is also known to use spectrophotometric methods, requiring the development of suitable chromogenic peptide reagents. Such methods provide a continuous precise assay for endopeptidases. Spectrophotometric methods, however, require relatively pure preparations of enzyme and are not normally suitable for evaluation of endopeptidase activities in crude or particulate samples.

Despite these efforts, at present, the primary convenient assay for the biological activity of the botulinum neurotoxins is the mouse lethality test. This test suffers from a number of drawbacks: (1) it is expensive and uses large numbers of laboratory animals, (2) it is non-specific unless performed in parallel with toxin neutralization tests using specific anti-sera, and (3) it lacks accuracy unless large animal groups are used. An improved cell-based assay is disclosed in US 2022/0010355 A1, the entire disclosure of which is hereby incorporated by reference herein. The present disclosure further improves on US 2022/0010355 A1 by providing anti-SNAP-25 antibodies that significantly reduce assay variability.

The purpose of the disclosed cell-based potency method is to determine the relative potency between botulinum toxins, such as BoNT/A run in the assay, replacing the LD₅₀ experiments on mice. In some aspects, the methods described herein determine the potency of BoNT/A, BoNT/E, and/or BoNT/C toxins, all of which are capable of cleaving SNAP-25. The methods described herein may be applied to any of the BoNTs described herein and their corresponding substrate cleavage partner.

LD₅₀ assays are highly process- and product-specific assays, which is exemplified through the lot-to-lot variability that can be seen in botulinum toxins, and the need for internal standardization for all manufacturers. This variability is further exemplified by the inability to standardize products and formulations from different manufacturers. LD₅₀ assays must be performed for every botulinum toxin composition. The nature of this assay also creates discrepancies, even when the same assay is performed in the same manner between batches. This is because the drug is cultured from bacterial fermentations, which inherently have variability between batches. Taken together, a skilled practitioner would be aware that one “unit” of on botulinum toxin preparation is not equivalent to another “unit” from a different composition unless proper LD₅₀ assay controls demonstrated potency equivalence. The methods described herein demonstrate the ability to determine relative potency between different samples, batches, products, and even different botulinum neurotoxin serotypes.

In some aspects, each assay utilizes three different toxin samples, categorized as Reference, Quality Control (QC), and Test. The Test sample has an unknown potency, the Reference is a sample with already established potency. A relative potency is calculated between the QC and Reference samples, and because this is a known value, it can be used as an Assay Acceptance Criteria. In some aspects, the QC sample is excluded and each assay utilizes two different toxin samples, the Reference sample and Test sample.

In some aspects, the reference sample comprises 10 U/ml, 20 U/ml, 30 U/ml, 40 U/ml, 50 U/ml, 60 U/ml, 70 U/ml, 80 U/ml, 90 U/ml, 100 U/ml, 110 U/ml, 150 U/ml, 200 U/ml, or 300 U/ml of the BoNT. In some aspects, the reference sample comprises about 10 U/ml, about 20 U/ml, about 30 U/ml, about 40 U/ml, about 50 U/ml, about 60 U/ml, about 70 U/ml, about 80 U/ml, about 90 U/ml, about 100 U/ml, about 110 U/ml, about 150 U/ml, about 200 U/ml, or about 300 U/ml of the BoNT. In some aspects, the reference sample comprises between 20 U/ml and 300 U/ml, 20 U/ml and 200 U/ml, 50 U/ml and 150 U/ml, 80 U/ml and 120 U/ml, 90 U/ml and 100 U/ml.

In some aspects, the QC sample comprises 10 U/ml, 20 U/ml, 30 U/ml, 40 U/ml, 50 U/ml, 60 U/ml, 70 U/ml, 80 U/ml, 90 U/ml, 100 U/ml, 110 U/ml, 150 U/ml, 200 U/ml, or 300 U/ml of the BoNT. In some aspects, the QC sample comprises about 10 U/ml, about 20 U/ml, about 30 U/ml, about 40 U/ml, about 50 U/ml, about 60 U/ml, about 70 U/ml, about 80 U/ml, about 90 U/ml, about 100 U/ml, about 110 U/ml, about 150 U/ml, about 200 U/ml, or about 300 U/ml of the BoNT. In some aspects, the QC sample comprises between 20 U/ml and 300 U/ml, 20 U/ml and 200 U/ml, 50 U/ml and 150 U/ml, 80 U/ml and 120 U/ml, 90 U/ml and 100 U/ml.

In some aspects, the test sample is found to have a potency of between 80-130% of the reference sample. In some aspects, the test sample is found to have a potency of between 80-130%, 80-120%, 80-110%, 80-100%, 80-90%, 90-130%, 90-120%, 90-110%, 90-100%, 100-130%, 100-110%, 110-130%, 110-120%, or 120-130% of the reference sample. In some aspects, the test sample is found to have a potency of within 1%, 2%, 3%, 4%, 5%, 6%, 7%, 8%, 9%, 10%, 15%, 20%, 25%, 30%, 35%, 40%, 45%, or 50% of the reference sample. In some aspects, the test sample is found to have a potency of within about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% of the reference sample.

In some aspects, the potency can be determined between 8-200 U/mL. In some aspects, the potency can be determined between 30-200 U/mL. In some aspects, the potency of a sample with greater than 200 U/mL can be determined by diluting the test sample by a factor or 1, 2, 5, 10, or 100, followed by determining the potency of each of each dilution and calculating the potency in view of the dilution factor utilized in diluting the sample. In some aspects, the potency of a sample with greater than 200 U/mL can be determined by serially diluting the test sample by a factor or 1, 2, 5, or 10 in 1, 2, 3, 4, 5, 6, 7, 8, 9, or 10 serial dilutions, followed by determining the potency of each of each dilution and calculating the potency in view of the dilution factor utilized in diluting the sample.

In some aspects, the potency of one or more BoNT products or samples are determined relative to a control sample for which the potency of each of the BoNT products are determined relative to the control sample. In some aspects, the potency of at least 2, 3, 4, 5, 6, 7, 8, 9, or 10 different BoNTs or BoNT samples are determined in parallel. In some aspects, the at least 2, 3, 4, 5, 6, 7, 8, 9, or 10 different BoNTs or BoNT samples are determined in parallel relative to a control sample, yielding a relative potency.

### A. Cell Culture

For this potency method, the iCells Motor Neurons (iPSC) from Cellular Dynamic International can be used, but other motor neurons can be suitable as well. The cells are generally stored at approximately −150° C., in thaw and use format.

The first step is to thaw and seed the cells into 96-well plates. The cells are thawed using a water bath set to 37° C. After thawing, the cells are diluted in cell media supplemented with the nutrients necessary for the cells' survival. Before adding the cells to the cell plates, the plates are coated with Poly-D-Lysine (PDL) and topped with Geltrex Matrix. This promotes adherence of the cells. The cells are then seeded to the plate by using a multichannel pipet.

From early experiments it was shown that there is an “edge effect” that affects the well-being of the cells, resulting in less healthy cells in the outermost wells of the plate. Therefore, the cells are seeded in the wells in a so-called “inner 60 well” format. One assay consists of three cell plates.

In order for the motor neurons to thrive, a media exchange is performed generally every two to three days after seeding. In this assay 75% of the media is exchanged on day 2, 5 and 7 after seeding. In some aspects, a non-proliferation agent is added to the cells. In some aspects, the non-proliferation agent exhibits γ-secretase. In some aspects, the non-proliferation agent is DAPT. During this first week, the media contains an agent called DAPT, which keeps the cells from proliferating into fibroblasts instead of motor neurons.

On day 9 after seeding, 50% of media is exchanged and the new media is from now on without DAPT. On day 12 after seeding the cells are treated with toxin.

In some aspects, the cells are subjected to a media exchange at least once per day post-seeding. In some aspects, the cells are subjected to a media exchange at least once every two days post-seeding. In some aspects, the cells are subjected to a media exchange at least once every three days post-seeding. In some aspects, the cells are subjected to a media exchange at least once every four days post-seeding. In some aspects, the cells are subjected to a media exchange no more than 2, 3, 4, or 5 times within a two day span post-seeding.

In some aspects, the media exchange is a 10% media exchange. In some aspects, the media exchange is a 20% media exchange. In some aspects, the media exchange is a 25% media exchange. In some aspects, the media exchange is a 30% media exchange. In some aspects, the media exchange is a 40% media exchange. In some aspects, the media exchange is a 50% media exchange. In some aspects, the media exchange is a 60% media exchange. In some aspects, the media exchange is a 70% media exchange. In some aspects, the media exchange is a 75% media exchange. In some aspects, the media exchange is an 80% media exchange. In some aspects, the media exchange is a 90% media exchange. In some aspects, the media exchange is a 100% media exchange.

In some aspects, the plates in which the cells are seeded into are tissue culture plates. In some aspects, the plates in which the cells are seeding into are coated with a substance that promotes cellular adhesion of the cells to the plates. In some aspects, the plates are selected from 4-well, 8-well, 12-well, 16-well, 24-well, 48-well, 96-well, 384-well, or 1536-well plates. In some aspects, the cells adhere to the plates. In some aspects, the cells do not adhere to the plates. In some aspects, the cells are adhered to or attached to the bottom of the wells of the plates. In some aspects, the cells are adhered to or attached to the bottom and the sides of the wells of the plates.

In some aspects, iCells Motor Neurons (iPSC) from Cellular Dynamics International or other motor neurons are utilized for the purpose of performing potency testing on them. The motor neurons can be population of human neurons derived from induced pluripotent stem cells. In some aspects, the cells grow on PDL+Geltrex matrix coated culture vessels, with media supplemented with DAPT replacement every 2-3 days for the first week and then in media without DAPT replaced every 2-3 days the rest of the culture time. The motor neurons are adherent cells and remain viable for up to or more than 14 days, which are generally stored in-150° C. low temperature freezers.

In some aspects, motor neurons are cultured utilizing one or more of the following media and/or supplements: iCell Neural Base Medium, iCell Neural Supplement A, iCell Nervous System Supplement, poly-D-lysine, Geltrex Basement Membrane Matrix, DAPT (≥98%), DMSO (Hybrid Mac), sterile water, 70% ethanol, and 0.4% trypan blue solution.

### Preparation of PDL and Geltrex Matrix Cell Culture Vessels

In some aspects, motor neurons grow on a coating of Poly-D-Lysine (PDL) with a fresh layer of Geltrex Matrix on top. In some aspects, the plating is prepared the same day as the cells are thawed.

In some aspects, the PDL-Geltrex Matrix plates can be prepared as follows:
1. Add PDL solution in each well in sterile cell culture plates according to Table 3. In some aspects, pre-PDK coated cell culture vessels may be used.
2. Incubate the cell culture plates with the lid closed, at room temperature for at least 1 hour.
3. After incubation with PDL, completely aspirate the solution from each well. Rinse each well twice with sterile water, and aspirate completely. PDL is generally toxic and must be rinsed away.
4. Add the Geltrex Matrix to each well according to table above and cover with the lid. Incubate at room temperature in the safety bench for at least an hour. The Geltrex is aspirated immediately before the cells are added. Prevent the Geltrex surface from drying out.

TABLE 3
Volumes for preparation of plates.

| | Volume of PDL | Volume of water | Volume of |
|---|---|---|---|
| Culture Vessel | solution (mL) | rinse or PBS (mL) | Geltrex (mL) |
| 6-well plate | 1 | 2 | 1 |
| 12-well plate | 1 | 2 | 0.8 |
| 24-well plate | 0.5 | 1 | 0.5 |
| 96-well plate | 0.1 | 0.2 | 0.1 |

### Prepare Motor Neuron Medium

In some aspects, the cell medium for Motor Neurons is supplied by Cellular Dynamics International together with the cells and consists of iCell Neural Base Medium (1 bottle, ˜100 mL), iCell Neural Supplement A (1 vial with ˜2 mL) and iCell Nervous System Supplement (1 vial with ˜1 mL). When prepared utilizing sterile techniques, the complete medium is then stable for 2 weeks when stored at 4° C.

Motor Neurons grow in complete medium supplemented with DAPT the first 9 days, and on subsequent media changes 50% of the DAPT-containing media is replaced with just complete media for the remaining of the culture time. In some aspects, the media is prepared as follows:
1. Thaw the supplements overnight at 4° C. or at room temperature protected from light for 30 min until no visible ice is in the tube.
2. Add the entire content of the supplements to the iCell Neural Base Medium bottle.
3. Rinse the supplement tubes with ˜1 mL medium each.
4. To make medium that can be used during the first week of culture, move 50 mL of the complete maintenance medium to a sterile 50 mL centrifuge tube. Write the date on the medium bottle and store in the fridge protected from light.
5. Dissolve DAPT in DMSO to achieve a concentration of 20 mM (8.6 mg/mL) by calculating the volume of sterile DMSO that needs to be added to the vial of DAPT. In some aspects, DAPT inhibits differentiation of hIPSC.
6. To the 50 mL tube of medium, add 12.5 μl of DAPT to achieve a final concentration of 5 μM.
7. Sterile filter this through a 0.22 μm sterile filter unit. Store at 4° C. protected from light.
8. The medium is to be equilibrated to room temperature when thawing cells as well as for media changes.

### Thawing and Plating Cells

In some aspects, cells are to be plated at approximately 1×10⁵ viable cells/cm². In some aspects, the cells are plated at about 1×10⁴, 2×10⁴, 4×10⁴, 6×10⁴, 8×10⁴, 1×10⁵, 2×10⁵, 4×10⁵, 6×10⁵, or 8×10⁵ viable cells/cm².

Table 4 provides exemplary dell densities and plating volumes for motor neurons.

TABLE 4
Cell densities and plating volumes for motor neurons.

| | | | Plating | | | |
|---|---|---|---|---|---|---|
| Culture | Surface area | | Volume | | Cell number | Cell number |
| Vessel | (cm²) | | (mL) | | (cells cm²) | (cells/mL) |
| 6-well plate | 9.6 | cm² | 2 | mL | 9.6 × 10⁵ | 4.2 × 10⁴ |
| 12-well plate | 3.8 | cm² | 1 | mL | 3.8 × 10⁵ | 1.25 × 10⁵ |
| 24-well plate | 2 | cm² | 0.5 | mL | 2.5 × 10⁵ | 2.5 × 10⁵ |
| 96-well plate | 0.32 | cm² | 0.2 | mL | 3.2 × 10⁴ | 6.25 × 10⁵ |

In some aspects, the cells are counted once thawed and added to the plates for culturing.

### Maintaining Cells

In some aspects, the cells, including motor neurons, can be maintained at least for 2 weeks. The first week, use Complete Maintenance Medium+DAPT and change 75% every 2-3 days. After 1 week of culture, perform 50% medium exchange with only Complete Maintenance Medium every 2-3 days.

In some aspects, neuronal cell lines are utilized in the methods described herein. In some aspects, the neuronal cell lines are human neuronal cell lines. In some aspects, the neuronal cell lines are mammalian neuronal cell lines. In some aspects, the neuronal cell lines are non-human cell lines. In some aspects, the neuronal cell lines are selected from the following: motor neuron, astrocyte, astroglia, neuroblast, dopaminergic neuron, cortical neuron, and neuron. In some aspects, the neuronal cell lines are motor neurons, interneurons, or sensory neurons.

In some aspects, the methods described herein utilize cells that express SNAP-25. In some aspects, the methods described herein may utilize non-neuronal cell lines that express SNAP-25. In some aspects, the cells natively express SNAP-25. In some aspects, the cells are modified to express a heterologous SNAP-25. In some aspects, the SNAP-25 is wild type. In some aspects, the SNAP-25 is modified.

### B. Toxin Treatment and Protein Extraction

In some aspects, toxin-treatment is performed on one cell plate at a time. In some aspects, the first step when toxin-treating the cells, is to collect 65% of the cell media from the culture wells. New media is added to the collected media and the mix is used for toxin dilution. The media collected from the culture is necessary to keep, since it contains vital substances created by the cells and 100% new media for toxin treatment would damage the cells. The cell media suspension is added to a 96-well mixing plate, mimicking the setup of the 96-well plate with the culture vessels. In the mixing plate the toxin samples are added to the first column of each row, and the toxin is then serial diluted throughout all the media filled columns. The rows which the reference, QC and unknown toxin units are added to are alternated over the three plates in the assay. When the dilution is finished, the remaining cell media in the culture vessels is discarded and replaced by the toxin containing media, column by column.

In some aspects, after toxin addition, the cells are incubated for 48 hours, allowing the toxin to exert its effect before the protein is extracted.

In some aspects, the cells are incubated with the toxin for at least 4 hours, at least 12 hours, at least 16 hours, at least 20 hours, at least 24 hours, at least 28 hours, at least 32 hours, at least 36 hours, at least 40 hours, at least 44 hours, at least 48 hours, at least 52 hours, at least 56 hours, at least 60 hours, at least 64 hours, at least 68 hours, or at least 72 hours.

In some aspects, the cells are incubated with the toxin for 4-96 hours, 24-96 hours, 48-96 hours, 4-72 hours, 4-48 hours, 4-24 hours, 24-96 hours, 24-72 hours, 24-48 hours, 48-96 hours, 48-72 hours, 36-56 hours, or 44-52 hours.

In some aspects, the cells are lysed by mechanical perturbation. In some aspects, the cells are lysed with a chemical that disrupts cell walls. In some aspects, the cells are lysed with a detergent. In some aspects, the lysed cells are separated from the resulting supernatant by centrifuge. In some aspects, the lysed cells are separated from the resulting supernatant by filtration.

In some aspects, during protein extraction all cell media is discarded from the cell plate. A cell lysis buffer called RIPA is then added to the wells, causing the cells to lyse, liberating the proteins. The RIPA buffer is supplemented with a protease inhibitor, to keep the proteins intact. After the addition of the cell lysis buffer, the cell plates are agitated to make the cells detach from the bottom of the dish. Finally, the lysate is transferred to a PCR-plate for storage in −20° C.

### Preparations

In some aspects, serial dilution of a botulinum neurotoxin, such as a BoNT type A, is required. In some aspects, the BoNT (e.g., BoNT A) may be formulated in a liquid formulation with a nominal concentration of 200 Units/ml (U/ml) or less.

In some aspects, the BoNT has a nominal concentration of 20 U/mL, 25 U/mL, 30 U/mL, 35 U/mL, 40 U/mL, 45 U/mL, 50 U/mL, 55 U/mL, 60 U/mL, 65 U/mL, 70 U/mL, 75 U/mL, 80 U/mL, 85 U/mL, 90 U/mL, 95 U/mL, 100 U/mL, 110 U/mL, 120 U/mL, 140 U/mL, 150 U/mL, 160 U/mL, 170 U/mL, 180 U/mL, 190 U/mL, 200 U/mL, 210 U/mL, 220 U/mL, 230 U/mL, 240 U/mL, or 250 U/mL.

In some aspects, the BoNT has a nominal concentration of about 20 U/mL, about 25 U/mL, about 30 U/mL, about 35 U/mL, about 40 U/mL, about 45 U/mL, about 50 U/mL, about 55 U/mL, about 60 U/mL, about 65 U/mL, about 70 U/mL, about 75 U/mL, about 80 U/mL, about 85 U/mL, about 90 U/mL, about 95 U/mL, about 100 U/mL, about 110 U/mL, about 120 U/mL, about 140 U/mL, about 150 U/mL, about 160 U/mL, about 170 U/mL, about 180 U/mL, about 190 U/mL, about 200 U/mL, about 210 U/mL, about 220 U/mL, about 230 U/mL, about 240 U/mL, or about 250 U/mL.

In some aspects, methods are designed for treatment of a 96-well plate where the edge wells are excluded, yielding a so called inner 60 well design. The dilution scheme is quarter log serial dilution, with two rows for a reference (REF) sample, two rows for a quality control (QC) sample and two rows for a Test sample.

In some aspects, the botulinum toxin is quickly deactivated using 0.2 M NaOH or hypochloride solution, such as ProChlor. These work by degrading the toxin.

Remove toxin vial stored at +4° C. and place it in room temperature 30 min prior to treatment or 1 h prior to treatment if stored at −80° C. Store the vials in dark and handle them with nitrile gloves on. Thaw a 15 mL aliquot of DP-buffer in room temperature 1 h prior to treatment. Remove cell medium from +4° C. and let it equilibrate to room temperature in dark for 30 min.

### Toxin Dilutions

### Option 1

Put six tips onto an 8-channel pipette and collect 130 μL cell medium from each well in the cell plate to a reservoir (total of 7.8 mL). Move the medium to a 50 mL falcon tube and add 13 mL fresh medium to it. Mix by turning the tube upside down twice. Put six tips onto the 8-channel pipette and add 300 μL cell medium to nine columns (column 1-9; 6 wells each) in the storage plate. Add additional 179.2 μL medium to column 1.

Open the vial of toxin with the decapper. If toxin contaminates the outer latex/vinyl gloves when removing the lid, change to a new pair. Pour DP-buffer in a reservoir. Before adding the toxin, set the single channel pipette to 200 μL and pre-wet the pipette tip five times with DP-buffer. Be careful to not let any DP-buffer remain in the tip before pipetting toxin. Thereafter, pipette the toxin up and down once and then add 200 μL to the correct well in column 1 on the storage plate. Set another single channel pipette to 5.2 μL and repeat the procedure.

Put six tips on an 8-channel pipette and set it to 300 μL, pre-wet the tips with DP-buffer five times and mix column 1 ten times. Lift 300 μL from column 1 to column 2 and repeat the procedure. Lift from column 2 to column 3, mix, and repeat until all wells are diluted.

### Option 2

Put six tips onto an 8-channel pipette and collect 130 μL cell medium from each well in the cell plate to a reservoir (total of 7.8 mL). Move the medium to a 50 mL falcon tube and add 13 mL fresh medium to it. Mix by turning the tube upside down twice. Mix 70% cell medium with 30% DP-buffer by adding 90 μL DP-buffer and 210 μL cell medium in column 2-9. To column 1, add 479.2 μL cell medium.

Open the vial of toxin with the decapper. If toxin contaminates the outer latex/vinyl gloves when removing the lid, change to a new pair. Pour DP-buffer in a reservoir. Before adding the toxin, set the single channel pipette to 200 μL and pre-wet the pipette tip five times with DP-buffer. Be careful to not let any DP-buffer remain in the tip before pipetting toxin. Thereafter, pipette the toxin up and down once and then add 200 μL to the correct well in column 1 on the storage plate. Set another single channel pipette to 5.2 μL and repeat the procedure.

Put six tips on an 8-channel pipette and set it to 300 μL, pre-wet the tips with DP-buffer five times and mix column 1 ten times. Lift 300 μL from column 1 to column 2 and repeat the procedure. Lift from column 2 to column 3, mix, and repeat until all wells are diluted.

In some aspects, the toxins are serially diluted by a factor of 10 for each dilution. In some aspects, the toxins are serially diluted by a factor of 5 for each dilution. In some aspects, the toxins are serially diluted by a factor of 2 for each dilution.

### Toxin Treatments and Incubation

In some aspects, all cell medium can be removed from one column in the cell plate the toxin dilution can be added. The cell plates fcan be incubated for 48 h in the incubator (95% O₂; 5% CO₂).

Cell Lysis and Protein Extraction from Cell Culture

In some aspects, the steps for protein extraction from cells can be carried out at 2-8° C. Dissolve one tablet protease phosphatase inhibitor per 10 mL RIPA buffer in a falcon tube. Carefully discard the medium in the cell culture plates with an 8-channel pipette. Add 120 μL ice cold RIPA buffer supplemented with inhibitor. Agitate the contents at 200 rpm for 30 min and at 4° C. Pipette the solution up and down ten times (avoid bubbles), collect the protein in fresh PCR-plates and directly store samples in −20° C.

### C. Determining Amount of Cleaved and Uncleaved SNAP-25 Proteins

In some aspects the Western blot is used as a tool for visualization of proteins in order to quantify the ratio between the cleaved and uncleaved SNAP-25 proteins in the protein samples from the cell cultures. The ratio between the proteins is used to calculate the EC50 values and thereby the potencies of the reference, QC and unknown toxin units, as described in the section Data analysis. The Western blot technique can roughly be divided into six parts, namely; sample preparation, gel electrophoresis, transfer, antibody incubation, imaging and analysis. The sample preparation is to prepare the proteins for separation by gel electrophoresis. After the separation the proteins are transferred, or blotted, from the gel to a membrane. The membrane is then incubated with antibody to enable the detection of the proteins of interest, after which the membranes are imaged and then analyzed through densitometric quantification.

The frozen protein samples are thawed and then prepared for separation by denaturation through addition of sample buffer and heat appliance. The sample buffer includes sodium dodecyl sulfate (SDS), which denaturizes the proteins and give them a net negative charge and 2-mercaptoethanol that reduces the intra- and intermolecular bonds and breaks disulfide bonds, making the proteins lose their tertiary structure. By denaturing the proteins, it is ensured that they have a similar charge to mass ratio and structure, so that they will be separated exclusively by their size in the gel electrophoresis.

The denaturized samples are loaded into wells on the top of TGX protein gels. Every gel contains 15 separate wells, all connected to a lane leading through the gel. The protein samples are loaded row-wise from high to low toxin treatment, onto the gels. Each gel holds 15 samples, and so all ten samples from the first row in the protein plate are loaded on the first gel, together with the five first samples from the second row. Then the last five samples from the second row are loaded onto the second gel, together with all ten samples from the third row, and so on. Hence, twelve gels are needed for one complete assay.

After separation of the proteins by gel electrophoresis, the proteins are transferred to membranes, i.e., blotted. In order to visualize the proteins two antibodies are used; a primary antibody that only recognizes whole and cleaved SNAP-25, and a secondary antibody that recognizes the primary antibody, enabling detection. In some embodiments, the primary antibody is directly labeled with a horse radish peroxidase enzyme using techniques known to those skilled in the art, obviating the need for the secondary antibody. Before applying the primary antibody, the membrane needs to be blocked using blocking buffer to reduce the unspecific binding of the antibody. After blocking, the primary antibody, dissolved in blocking buffer, is applied and the membrane is incubated overnight to allow the antibody to attach to the proteins. The membrane is then washed using a pH-stable buffer, to remove all excessive antibodies, before application of the secondary antibody. After incubation with the secondary antibody, the membrane is washed again before development.

When developing the images of the proteins, the membranes are incubated with a luminol/peroxidase substrate. The secondary antibody is conjugated with a horse radish peroxidase enzyme (HRP) that catalyzes an oxidation reaction between luminol and peroxidase, resulting in luminol emitting light, a chemiluminescence. As multiple secondary antibodies may bind the primary antibody, amplification of signal and increased sensitivity may be achieved. By using a CCD camera, the emitted light is detected, with the intensity corresponding to the amount of protein present. In some embodiments, emitted light is detected using auto exposure in a biorad ChemiDoc XSR+ imager. A representative image obtained from a western blot membrane is shown in FIG. 1. In some embodiments, the antibody is directly HRP labeled (FIG. 3), thereby reducing variability in protein detection.

In some aspects, the methods described herein require the separation of cleaved SNAP-25 from uncleaved SNAP-25 for comparison purposes. In some aspects, the identification of cleaved from uncleaved SNAP-25 is performed via capillary (gel-free) Western blotting, native gel electrophoresis followed by immunoblotting, denatured gel electrophoresis followed by standard Westering blotting, 2D gel electrophoresis followed by immunoblotting, and microscale Western blotting.

In some aspects, alternatives to Western blots may be utilized to determine the ratio of cleaved SNAP-25 to uncleaved SNAP-25, such as microscale Western blot (U.S. Pat. No. 9,182,371) or gel-free Western blot (U.S. Pat. No. 9,523,684). In some aspects, quantifying the cleaved and uncleaved SNAP-25 is performed via antibody capture of the cleaved and uncleaved SNAP-25, purifying the antibody captured proteins via affinity purification and/or HPLC and further determining the amount of cleaved and uncleaved SNAP-25.

Detecting SNAP-25 Protein with Western Blot (WB)

In some aspects, WB is utilized as a detection method for a cell based assay (CBA) to detect the SNAP-25 protein (both cleaved and non-cleaved forms) after treatment with botulinum neurotoxin A in cell cultures.

In some aspects, the WB technique can roughly be divided into six parts; sample preparation, gel electrophoresis, transfer, antibody incubation, imaging and analysis. The sample preparation is to prepare the proteins for separation by gel electrophoresis. After the separation the proteins are transferred, or blotted, from the gel to a membrane. The membrane is then incubated with antibody to enable the detection of the proteins of interest, after which the membranes are imaged and then analyzed through densitometric quantification. After imaging the membranes are discarded.

In some aspects, each potency determination generates three cell plates with 60 protein samples apiece that are to be detected using WB. The gels used for the separation holds 15 protein samples, and thus 12 gels are needed for one complete assay (four for each cell plate). In order to avoid thawing of the samples several times, all samples on one cell plate should preferably be run at the same time. The following method generally describes the procedure for running one gel. However, all amounts of solutions can be multiplied if more cell plate samples are run in one day.

### Electrophoresis (Day 1)

Sample preparation and gel electrophoresis. In some aspects, the steps involve samples containing 2-Mercaptethanol, including gel electrophoresis, are performed in a ventilated hood. The wells in the gel can contain 15 μl/well, however, a total volume of 10 μl/well is used.
1. Switch on the heat-block to 95° C. and thaw the frozen protein samples on ice for about 45 min.
2. While the protein samples are thawing, prepare the sample buffer and the running buffer:
2a. Start by labeling the Eppendorf tubes, one for each sample that will be run, including one for the sample buffer. 15 samples are run on each gel and four gels at a time.
2b. Prepare sample buffer in a ventilated hood by adding 2-Mercaptoethanol to 2× Laemmli Sample Buffer in volumes indicated in Table 5 below into the designated Eppendorf tube, and make sure to mix well by pipetting up and down 5 times using a 100 μl pipet. Add 4 μl sample buffer to each Eppendorf tube labelled for protein samples. \
2c. Prepare the Running Buffer according to Table 6. Approximately 750 ml Running Buffer in each tetra cell is used. The running buffer is kept in room temperature.

TABLE 5
Volumes used for sample buffer preparation.

Sample Buffer preparation

| Number | Total volume | 2x Laemmli | 2-Mercaptoethanol |
|---|---|---|---|
| of gels | (μl) | (μl) | (μl) |
| 2 | 150 | 142.5 | 7.5 |
| 4 | 300 | 285 | 15 |
| 6 | 400 | 380 | 20 |
| 8 | 500 | 475 | 25 |
| 12 | 780 | 741 | 39 |

TABLE 6
Running buffer preparation.

Running Buffer preparation

| Number | Total Volume | Deionized water | 10x Running |
|---|---|---|---|
| of gels | (mL) | (mL) | buffer (mL) |
| 2 | 1000 | 900 | 100 |
| 4 | 2000 | 1800 | 200 |
| 6 | 3000 | 2700 | 300 |
| 8 | 4000 | 3600 | 400 |
| 12 | 5000 | 4500 | 500 |

3. When the protein samples are thawed, pipette the protein samples up and down five times, using a 100 μl 12 channeled multi pipet for one row at the time, to mix the protein sample before transferring from cell plate. Add 6 μl of protein sample in the Eppendorf tubes.
4. Incubate the samples for 5 min at 95° C. in a heat-block, to denature the proteins.
5. Meanwhile, prepare the gels.
5a. Open the gel packages and remove the green tape on the bottom of each gel before inserting them into the chamber. If only one gel is used, use the artificial plastic gel to close the chamber.
5b. Fill the inner gel chamber to the edge with running buffer. Make sure that the gel chamber is sealed tight and that no buffer is leaking. Then, add running buffer to the marking for 2 gels on the Tetra Cell. The gels can also be prepared this far while the protein samples are thawed.
5c. Remove the green plastic comb on top of the gels and use a Pasteur pipet to wash the wells two times.
6. Take the samples from the heating block. Pipette the samples up and down twice from the bottom of the Eppendorf tube). The 10 μl of the protein samples are loaded row-wise from high to low toxin treatment. Each gel holds 15 samples, and so all ten samples from the first row in the plate are loaded on the first gel, together with the five first samples from the second row. Then the last five samples from the second row are loaded onto the second gel, together with all ten samples from the third row, and so on. No ladder is used.
7. Place the top in the gel chamber and run the electrophoresis 150 V for 90 min. Make sure that the electrophoresis starts by checking for bubbles emerging from the bottom of the gel cassette.
8. Prepare the Blocking Buffer according to Table 7. The solution is kept in room temperature until the first blocking step, then at 4° C.

TABLE 7
5% blocking buffer preparation.

| Number | Amount of 5% Blocking Buffer |
|---|---|
| of gels | (Blocking Buffer, 1XTTBS) |
| 2 | 100 ml (5 g, 100 ml) |
| 4 | 150 ml (7.5 g, 150 ml) |
| 6 | 250 ml (12.5 g, 250 ml) |
| 8 | 300 (15 g, 300 ml) |
| 12 | 500 (25 g, 500 ml) |

### Transfer (Day 1)

1. Prepare for transfer before the electrophoresis is done by taking out the Trans-Blot Turbo Transfer Packs needed from the fridge, one pack is needed per gel. Also get the spatula, rolling pin and the scalpel blade. Take out a cassette from the Trans Blot Turbo Transfer System and remove the top. When the electrophoresis is finished, take out the tetra cell from the hood, detach the gel cassettes and put them pack in the hood.
2. Put the Trans Blot Turbo Transfer pack and cassette in the ventilated hood. Open the transfer pack from the upper right corner and take out the “bottom”-stack, using your hand, and put it in the transfer cassette. Do not invert the stack when moving it to the cassette, lift it as it is in the transfer pack. The membrane is on top of the bottom stack, so try to touch it as little as possible. Use the spatula to break the plastic cover around the gel, where to put the spatula is marked with arrows. Cut off additional gel residues at the top and bottom of the gel using the scalpel blade. Align the bottom of the wells at the top and the dark front line at the bottom of the gel cassette. Use a rolling pin to remove any air-bubbles from the stack-gel composition. Close the cassette with the top cassette. Make sure the top cassette is in “locked” position. The package for the transfer pack is discarded in a waste bin.
3. Use the program TGX Turbo Transfer in the Turbo Transfer System. Insert the cassette in the upper (A) or lower (B) bay. Switch on the Turbo Transfer System. On the home screen, chose the “Turbo” protocol, then chose “1 MINI TGX”. The protocol will be set for a 3 min run with 2.5 A and 25V. Chose A- or B-run, depending on in which bay the cassette is in, to start the run. When the run is complete, the proteins have been transferred to the PVDF membrane.
4. After transfer, the membrane is pre-blocked in 15 ml of blocking buffer for 1 h at agitation (30 rpm, 05 degrees angle) in RT in a small plastic chamber.
4a. Start by pouring the 15 ml of blocking buffer in the plastic chamber.
4b. Take off the top cassette and discard the top stack and the gel in a waste bin.
4c. Carefully lift the membrane with one hand and cut the top of the membrane to fit it into the chamber, the membrane should be free-floating. Also cut off the upper corner of the side of the membrane where the first well of the gel was blotted, to keep track of what is right and left and up and down of the membrane. However, touch the membrane as little as possible. If the transfer was successful, faint transparent traces of the wells/lanes in the gel is visible.
4d. Put the membrane in the blocking buffer when finished and put the plastic chamber on the shaker.
5. The bottom stack is then discarded in the waste bin. Wipe off remaining transfer buffer from the cassette using paper towels and clean the cassette with deionized water. Wipe off most of the water with paper towels and leave the cassette disassembled for drying for a couple of hours before it is inserted back into the Turbo Blot Transfer machine.

### Primary Antibody Incubation

After blocking, incubate the membrane with primary antibody for 10 minutes before the blocking is done, take out the blocking buffer from the fridge and the primary antibodies from the freezer. When the antibodies are thawed, after a few minutes in RT, dilute the primary antibodies in blocking buffer. Anti-SNAP-25 is diluted 1:1000 and anti-B-actin (A1978) 1:2000, in volumes given in Table 8. Gently vortex the diluted antibody for about two seconds. The anti-B-actin antibody may be skipped. It is not needed for the analysis, but can be included for troubleshooting the assay.

Cut one reaction folder in four, a quarter folder is enough for one membrane. Seal one side of the cut-out folder piece using the bag sealer.

When the blocking of the membrane is done, carefully lift the membrane into the prepared folder using tweezers. Put the membrane as close as possible to the sealed side of the folder. Avoid dragging the membrane along the plastic. Seal two more sides of the plastic folder using the bag sealer. Seal as close to the membrane sides as possible, without sealing on the membrane. Pour the diluted antibodies into the folder.

Carefully remove all large air-bubbles in the antibody solution using fingers. Avoid squeezing the membrane too hard. When no large bubbles are left (small air-bubbles that can easily move around are alright), seal the last side of the plastic folder with the bag sealer. Put the enclosed membrane on a shaker set to 100 rpm with infinity setting (no timer) at 4° C. Leave the membranes for overnight incubation.

TABLE 8
Primary antibody solution preparation.

| Number | Amount of Primary antibody solution |
|---|---|
| of gels | (anti-SNAP/anti-B-actin, Blocking Buffer) |
| 2 | 10 ml (10 μl/5 μl, 10 ml) |
| 4 | 20 ml (20 μl/10 μl, 20 ml) |
| 6 | 30 ml (30 μl/15 μl, 30 ml) |
| 8 | 40 ml (40 μl/20 μl, 40 ml) |
| 12 | 60 ml (60 μl/30 μl, 60 ml) |

### Secondary Antibody Incubation

Prepare a small plastic chamber for washing of the membrane by filling it with 20 ml of 1×TTBS.

Cut open the plastic bag with the membrane using scissors. Carefully lift the membrane with tweezers to the prepared plastic chamber. The membrane should be free-floating, otherwise add more 1×TTBS. Wash the membrane for 10 min in RT on agitation (30 rpm, 05 degrees angle).

After 10 minutes, gently pour off the 1×TTBS into a plastic chamber used for waste. Quickly refill the plastic chamber with 20 ml of 1×TTBS, repeat after 10 more minutes (in total 3×10 minutes of washing).

When the last wash has been started, prepare the secondary antibodies, according to Table 9. Use secondary antibodies diluted 1:10 000 in 5% Blocking Buffer. For one membrane, add 1.5 μl of both antibodies to 15 ml of blocking buffer.

TABLE 9
Secondary antibody solution preparation.

| Number | Amount of Secondary antibody solution |
|---|---|
| of gels | (anti-secondary/anti-secondary, Blocking Buffer) |
| 2 | 30 ml (3 μl/3 μl, 30 ml) |
| 4 | 60 ml (6 μl/6 μl, 60 ml) |
| 6 | 90 ml (9 μl/9 μl, 90 ml) |
| 8 | 120 ml (12 μl/12 μl, 120 ml) |
| 12 | 180 (18 μl/18 μl, 180 ml) |

Once the last wash is finished, pour off the 1×TTBS into the waste chamber and add the secondary antibody dilution to the plastic chamber with the membrane. Incubate the membrane in secondary antibody at RT on agitation (30 rpm, 05 degrees angle) for 1 h.

When the incubation with secondary antibody is finished, pour off the antibody solution into the waste chamber and add 20 ml of 1×TTBS to the chamber with the membrane. Wash the membrane in 1×TTBS for 3×10 min in RT on agitation (30 rpm, 05 degrees angle). Prepare for development during the last washing step.

### Development (Day 2)

During the last wash step, prepare the CLARITY Western ECL Substrate, 1:1 luminol enhancer: Peroxidase buffer, according to Table 10. For one membrane, add 3 ml of each solution to a 15 ml falcon tube (i.e., 6 ml substrate in total). Turn the falcon tube back and forth at least four times to mix the solution.

TABLE 10
Development solution preparation.

| Number | Amount of ClarityTM Western ECL |
|---|---|
| of gels | Substrate (Luminol, Peroxidase) |
| 2 | 12 ml (6 ml, 6 ml) |
| 4 | 24 ml (12 ml, 12 ml) |
| 6 | 36 ml (18 ml, 18 ml) |
| 8 | 48 ml (24 ml, 24 ml) |
| 12 | 72 ml (36 ml, 36 ml) |

When the last wash is finished, pour off the 1×TTBS to a waste chamber. Pour the Clarity™ Western ECL Substrate from the falcon tube onto the membrane. Make sure that the entire membrane is covered with substrate by checking for dry spots. Incubate the membrane in the substrate for 3 min. While the membrane is being incubated, prepare for capturing an image of the membrane and ultimately image the membrane. In some aspects, the membrane is captured for a duration of 0.5-30 seconds. In some aspects, the membrane is captured for a duration of two minutes.

### D. Densitometric Quantification of Gels and Analysis

In some aspects, the images obtained from the western blot are quantified using the software ImageLab. This is software developed for the usages together with the CCD camera, both from Bio-Rad. Using the ImageLab, the bands of proteins can be quantified depending on the intensity of each pixel in the bands. In this way a ratio may be obtained between the intensities of the bands showing uncleaved and cleaved SNAP-25. ImageLab calculates total volume of the bands in each lane, which are manually separated. In some aspects, from the total volumes the software gives a Band % that is used for EC50 calculation with GraphPad Prism. In some aspects, QuBase biostatistics software is used to for EC50 calculation. The Band % can be used for effect calculations since 100% effect corresponds to 100% cleaved SNAP-25. The EC₅₀ value is the concentration of the BoNT product at which half of the response is given and 50% of all SNAP-25 is cleaved.

In some aspects, the images obtained of the membranes from the western blot (WB) procedure are analyzed in order to calculate an EC₅₀ value for a given BoNT or BoNT product. The EC₅₀ value corresponds to the concentration of the BoNT or BoNT product that gives half of the response. In this case the EC50 value is decided as a 50/50 ratio between cleaved and uncleaved SNAP-25. Hence, a densitometrical quantification of the protein bands from the WB is used to determine the ratios of the proteins in each sample from the cells treated with various concentrations of the toxin.

In some aspects, this protocol describes how to perform a densitometric quantification using the software Image Lab. The generated data will then be further analyzed in the software GraphPad Prism or any similar performing software.

In some aspects, prior to beginning analysis, ensure that there are no overexposed bands among the bands that are to be analyzed. The intensity percentages may not be correct if overexposed bands are included in the analysis. Overexposed bands will be highlighted in red by the software. In some aspects, if there are red highlights in the image, choose a new image with a shorter exposure time.

In some aspects, in Image Lab, click on the button “Image tools” in the Analysis Tool Box (the left column of the starting window). If necessary, flip the membrane vertically or horizontally by clicking the corresponding button under the section “Flip” in the left column (i.e., “Vertical” or “Horizontal”). The highest toxin concentration should be to the left, to easier keep track of which lane that corresponds to a certain column from the cell plate that was analyzed. It is also possible to rotate the picture by clicking on the button “Custom” under the section “Rotate”. The protein bands should be as horizontal as possible, to simplify the analysis. After clicking “Custom”, grab the red arrow that appeared on the image and drag it to the right or left to the desired rotation; right click on the image and chose “Rotate” to implement the rotation. Click “Ctrl+Z” on the keyboard to redo any unsatisfactory changes.

In some aspects, in Image Lab, return to the Analysis Tool Box by clicking on the arrow on the left hand side of the “Image Tools” heading at the top of the left column in the starting window. Click on the button “Lane and Bands” in the Analysis Tool Box. Under the section “Lane Finder”, chose “Manual . . . ”. In the new window that opened, type in the number of lanes that are to be analyzed. Resize the frame for the lanes by grabbing and dragging the corners or sides of the frame. The frame should be adjusted so that all bands that are to be detected are completely within the outer lines of the frame.

In some aspects, when all bands are within the outer lines of the frame, click the “Adjust background . . . ”-button in the left column of the starting window. In the new window that opened set the Disk Size (in the section “Background Subtraction” at the bottom of the window) to 1.0 mm and click “Apply to all lanes.”

In some aspects, when all bands are fitted into the boxes (two bands in each box, the upper for uncleaved SNAP-25 and the lower for cleaved SNAP-25), chose the tab “Bands” at the top of the left column in the starting window. Under the section “Band Finder” click on the button “Detect Bands . . . ”

In some aspects, check in the “Advance” button under the section “Detection Settings” and “Band Detection Sensitivity”. Set the Sensitivity to 80.00; Size Scale to 3; Noise Filter to 3, and Shoulder to 6. These settings alter the resolution at which the bands are being detected. Then click on the “Detect”-button at the bottom of the “Band Detection” window.

In some aspects, it may be beneficial to remove any unwanted detection bands, i.e. if there are two bands created for a single protein band or if there are bands outside of the two bands that are to be analyzed. This is done by clicking on the “Delete” button in the left column of the starting window and then clicking on the bands that are to be removed. Bands can also be added by using the “Add” button. In this case, the limits for the existing bands might need to be adjusted using the “Adjust” button, to make it possible to add a band where wanted.

In some aspects, it might be necessary to adjust the limits of each detection band to ensure that the protein bands are quantified in the most correct manner. At the top of the starting window, click on the button “Lane Profile”. A new window will appear. Make sure that you have unmarked the “Detect”, “Add” or “Adjust” buttons from the previous step by clicking these icons again. Then click on the first lane box. In the “Lane Profile” window, the intensity of the protein bands is now shown as a curve, and it is possible to adjust the limits of the detection bands accordingly. Click on the blue lines beneath the curve plot and drag these sideways to adjust the limits. Make sure that all tops in the curve are fully within the limits, and following these guidelines:

Do not adjust the limits if not necessary. When there are two separate tops, make sure that the smaller top is fully within the limit if the default gap between the band limits does not fit between the curves. If the curves are not completely separated, adjust the limit of the smaller bulb so that the detection band is directly above the protein band. If a double band was removed, the limit of the band might be in the middle of the curve. Then adjust the limit to fit the entire curve, i.e., until the curve meets the background line.

When the limits have been satisfactorily set, the “Lane Profile” window may be closed. To retrieve the quantification of the bands, click on the button “Analysis Table” in the top bar of the starting window. The “Analysis Table” will appear at the bottom of the window. At the top of the “Analysis Table”, click on the button for changing the orientation of the analysis table, so that the data for each lane is shown on top of each other, as a vertical column instead of a horizontal line as is set as default. Then click on the button “Export analysis table to Excel” at the top of the “Analysis Table”. The band data will now be opened in an Excel file.

In the Excel data file there will now be one or two rows for each lane from the image analysis, one row for each protein band. If there are two rows, i.e., two bands, with the same lane number, the band number 1 will be for the uncleaved SNAP-25 and band number 2 for the cleaved SNAP-25. In the Excel file, copy the Band % for the cleaved bands to a new column to more easily copy the data for further analysis. Start by naming one column “Sample” and write down each sample that was analyzed, i.e. if the rows B and C from one plate were included on this image, name the rows B10, B9, B8, . . . , B1, C5, C4, . . . , C1. Then name the column next to the “Sample” column “% Cleaved” and copy the Band % for each sample to the corresponding row.

In some aspects, in the instance of only one band in the gel, go back to the image and check if it's a 100% cleaved or uncleaved band, and add 100 or 0 accordingly. The sample data are preferably added from low to high toxin concentration, to ease the transfer of the data to GraphPad Prism for analysis.

EC₅₀ Analysis with GraphPad Prism (v. 5.02)

In some aspects, the EC₅₀ is determined utilizing GraphPad Prism to visualize the data, as follows:

In some aspects, in order to analyze the data sets, click on Analyze and under XY analyses choose Nonlinear regression (curve fit), thereafter OK.

In some aspects, choose under Dose-response-Stimulation, Log (agonist) vs response-Variable slope (four parameters). Thereafter, click on the tab Constrain and choose Constant equal to 0 for bottom and for top Constant equal to 100. This will result in a constrained analysis. Click OK.

In some aspects, this will result in a table with absolute EC₅₀ values including standard Error. Relative potencies are calculated by dividing the EC₅₀ values for Test and QC samples with the reference sample. The standard error for the relative potency is calculated based on error propagation according to:

$$SE RP ( QC ) = RP ( QC ) * SQRT ( {( SE Ref / {EC}_{50} Ref )}^{2} + {( SE QC / {EC}_{50} QC )}^{2} )$$

$$SE RP ( Test ) = RP ( Test ) * SQRT ( {( SE Ref / {EC}_{50} Ref )}^{2} + {( SE Test / {EC}_{50} Test )}^{2} )$$

Where SE is standard error and RP is relative potency. Note, this calculation for relative potency is valid only if the three curves are paralell.

The graph is ploted automatically, and can be found under the Graphs sections in the left column. The X-axis may be formatted to go from −5 to +2 on the log scale while the Y axis may be formatted to 110-150, depending on the needs evidenced by the data.

EC₅₀ Analysis with R (v. 3.6.1)

In some aspects, the EC₅₀ analysis is performed with R through the dose response curves (DRC) package (3.0-1). See Ritz et al. (PLOS One. 2015 Dec. 30; 10 (12):e0146021. doi: 10.1371/journal.pone.0146021).

For DRC, a table of a specific format must be created that provides the raw data captured from the densitometry images of the experimental gels.

Start the R environment. At the prompt, enter the following:
library (drc) (This loads the DRC package)
setwd (“C:/DRC”) (This sets working directory to DRC)
source (“3plate.rs”) (This executes the script which performs the analysis)

### E. Statistical Analysis

In some aspects, statistical analysis of the data is performed with GraphPad Prism (v. 5.0). In some aspects, a 4 parametric logistic (4PL) curve is fitted to the data using a model constraining the curve between 0 and 100%.

In some aspects, statistical analysis of the data is performed in R (v. 3.6.1, 64 bit) utilizing methods implemented in the dose response curves (DRC) package (3.0-1). In some aspects, a 4PL curve is fitted to the data using an unconstrained model. In some aspects, errors are expressed as standard errors and error propagation may be used to estimate the error for relative potencies.

In some aspects, statistical analysis of the data is performed in QuBase biostatistics software.

### Snap-25 and BoNT Clevage of SNAP-25

“SNAP-25” refer to a peptide sequence, or fragment thereof, of a human SNAP-25 protein (Entrez 6616; UniProt P60880). SNAP-25 is a Target Soluble NSF (N-ethylmaleimide-sensitive factor) Attachment Protein Receptor (t-SNARE) protein encoded by the SNAP25 gene found on chromosome 20p12.2 in humans. SNAP-25 is a component of the trans-SNARE complex, which accounts for membrane fusion specificity and directly executes fusion by forming a tight complex that brings the synaptic vesicle and plasma membranes together, regulating neurotransmitter release. The amino acid sequence of human SNAP-25 is:

MAEDADMRNELEEMQRRADQLADESLESTRRMLQLVEESKDAGIRTLVML
DEQGEQLERIEEGMDQINKDMKEAEKNLTDLGKFCGLCVCPCNKLKSSDA
YKKAWGNNQDGVVASQPARVVDEREQMAISGGFIRRVTNDARENEMDENL
EQVSGIIGNLRHMALDMGNEIDTQNRQIDRIMEKADSNKTRIDEAN
QRATKMLGSG.

BoNT/A catalyzes the hydrolysis of the Gln197-Arg198 peptide bond, bolded above.

BoNTs bind specifically to the presynaptic surface of neurons that use the neurotransmitter acetylcholine. Once bound to the nerve terminal, the neuron uptakes the BoNT into a vesicle by receptor-mediated endocytosis. As the vesicle moves farther into the cell, it acidifies, activating a portion of the BoNT that triggers it to push across the vesicle membrane and into the cell cytoplasm. Once inside the cytoplasm, the toxin cleaves SNARE proteins, meaning that the acetylcholine vesicles cannot bind to the intracellular cell membrane, preventing the cell from releasing vesicles of neurotransmitter. This stops nerve signaling, leading to flaccid paralysis.

### EXAMPLES

The examples discussed below are intended to be purely exemplary of the invention and should not be considered to limit the invention in any way. The examples are not intended to represent that the experiments below are all or the only experiments performed.

### I. Example 1—SNAP-25 Binding and Detection Assessments

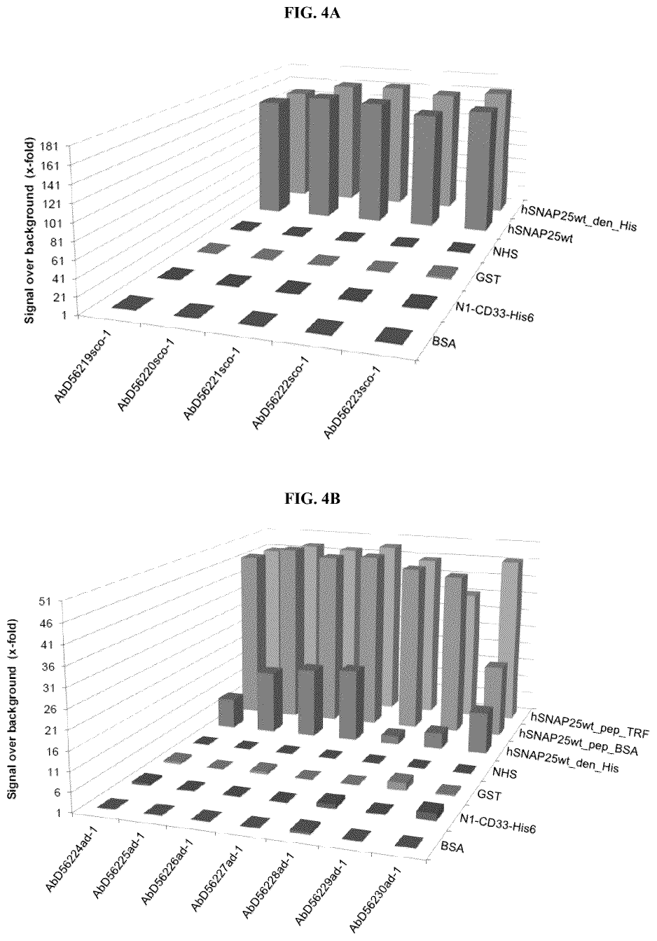

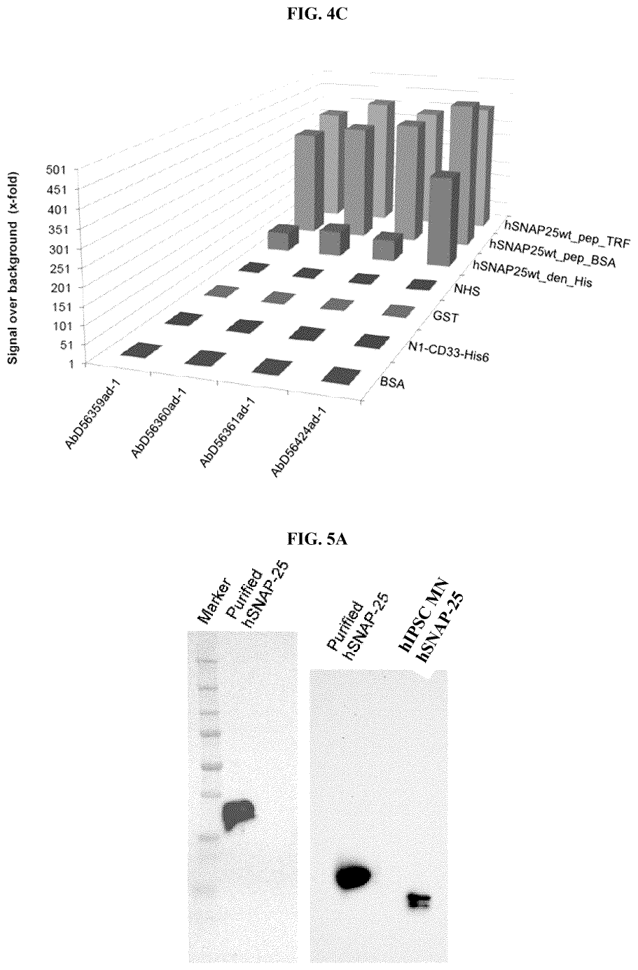

20 candidate recombinant Immunoglobulin G-based monoclonal antibodies were identified from a library of 45 billion antibodies. Candidate antibodies AbD56219sco-1, AbD56220sco-1, AbD56221sco-1, AbD56222sco-1, AbD56223sco-1 (FIG. 4A), AbD56224ad-1, AbD56225ad-1, AbD56226ad-1, AbD56227ad-1, AbD56228ad-1, AbD56229ad-1, AbD56230ad-1 (FIG. 4B), AbD56359ad-1, AbD56360ad-1, AbD56361ad-1, and AbD56424ad-1 (FIG. 4C) were assessed for their ability to bind purified human SNAP-25 in a direct ELISA assay. Binding was assessed using three positive antigens (hSNAP25 wt_pep_TRF, hSNAP25 wt_pep_BSA, and hSNAP25 wt_den_His) and four negative antigens (NHS, GST, Nf-CD33-His6, and BSA). Fold signal to background was calculated. From these results, 14 antibodies were selected for further evaluation in Western blot assays.

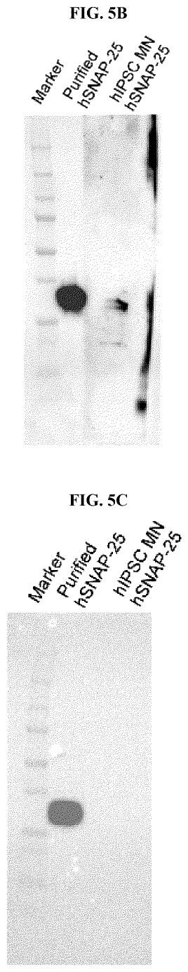

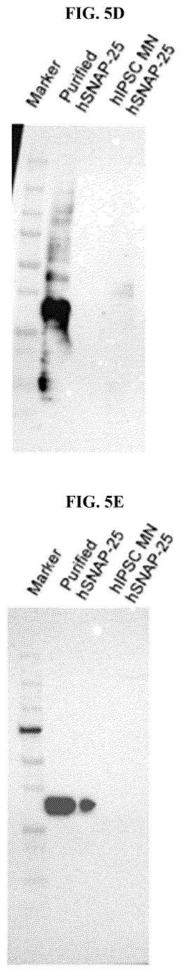

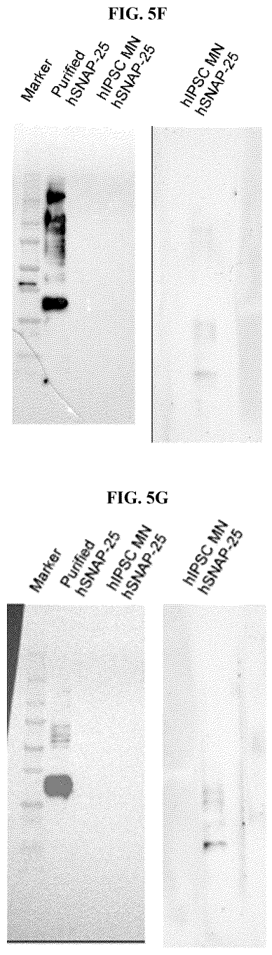

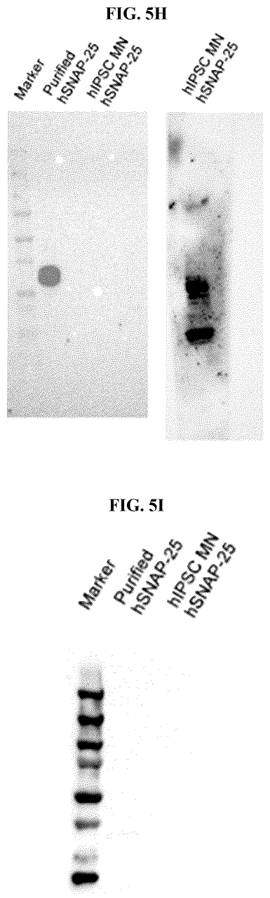

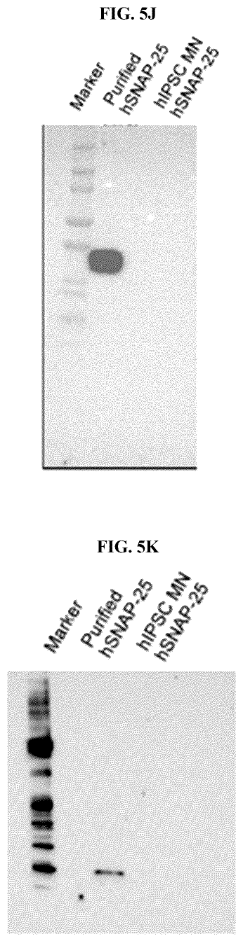

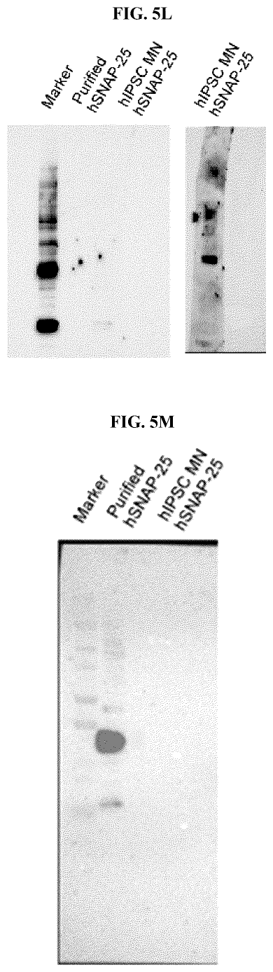

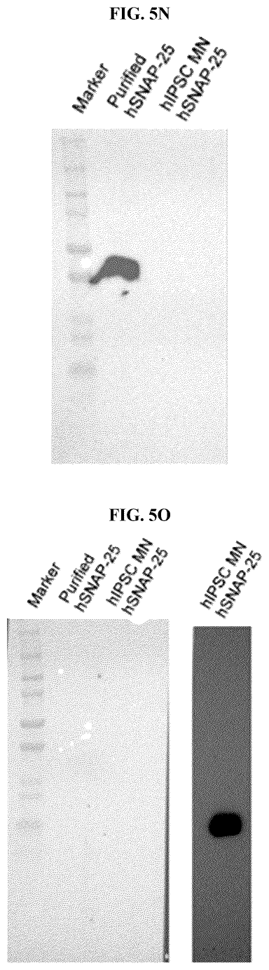

Further evaluation was performed with candidate antibodies AbD56219sco-1 (FIG. 5A), AbD56220sco-1 (FIG. 5B), AbD56221sco-1 (FIG. 5C), AbD56222sco-1 (FIG. 5D), AbD56223sco-1 (FIG. 5E), AbD56224ad-1 (FIG. 5F), AbD56225ad-1 (FIG. 5G), AbD56226ad-1 (FIG. 5H), AbD56227ad-1 (FIG. 5I), AbD56228ad-1 (FIG. 5J), AbD56229ad-1 (FIG. 5K), AbD56230ad-1 (FIG. 5L), AbD56360ad-1 (FIG. 5M), AbD56424ad-1 (FIG. 5N), and AbD56359ad-1 (FIG. 5O). Antibodies AbD56219sco-1, AbD56220sco-1, AbD56221sco-1, AbD56223sco-1, AbD56226sco-1, AbD56228sco-1, AbD56229sco-1, AbD56359sco-1 and AbD56424sco-1 specifically detect 10 ng purified SNAP-25, as can be seen in Western blots FIG. 5A, FIG. 5B, FIG. 5C, FIG. 5E, FIG. 5H, FIG. 5J, FIG. 5K, FIG. 5M, and FIG. 5N, respectively. Further, antibodies AbD56219sco-1, AbD56220sco-1 and AbD56230sco-1 detected native SNAP-25 from 3,000 hIPSC MNs, as can be seen in Western blots FIG. 5A, FIG. 5B, and FIG. 5N, respectively. Further, antibodies AbD56222sco-1, AbD56225sco-1, AbD56360sco-1 detected 10 ng purified SNAP-25 with low specificity, also detecting non-SNAP-25 proteins, as can be seen in Western blots FIG. 5D, FIG. 5G, and FIG. 5N, respectively. Further, antibodies AbD56222sco-1 and AbD56225sco-1 detected native SNAP-25 from 3,000 hIPSC MNs with low specificity, as can be seen in Western blots FIG. 5D and FIG. 5G, respectively.

It is noted that purified SNAP-25 has a higher molecular weight than native SNAP-25 due to fusion of HA, myc, and FLAG tags.

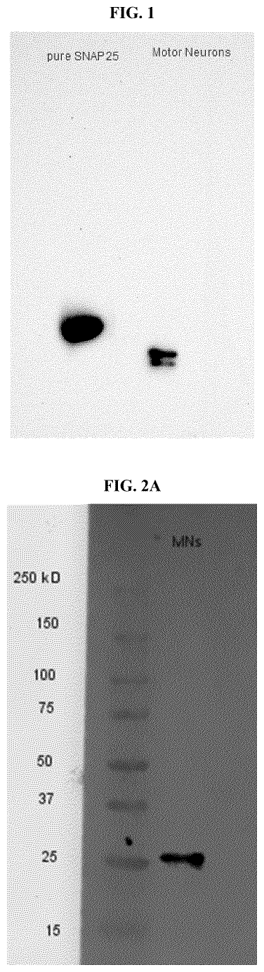

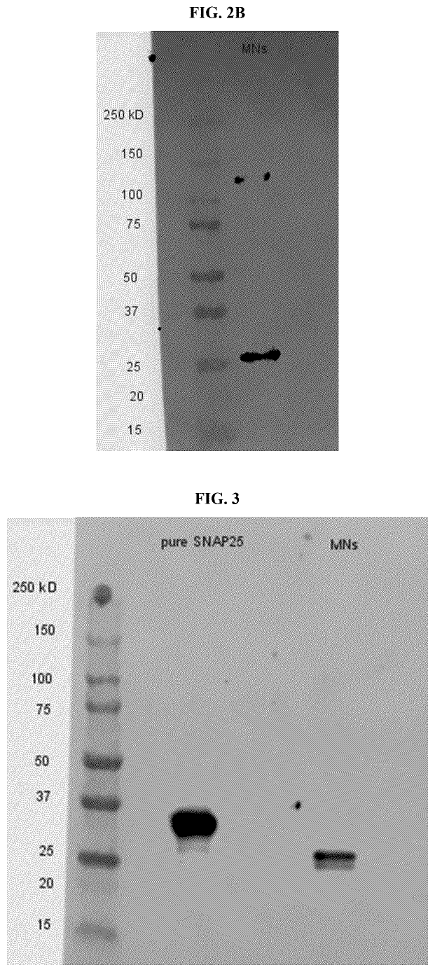

Based on these results, AbD56219sco-1 (FIG. 5A) was selected for further Western blot evaluations. AbD56219sco-1 detected 10 ng of purified SNAP-25 and SNAP-25 from 3,000 cultured hIPSC MNs exposed to 3 LD₅₀ U/ml of BoNT/A with high specificity at an antibody concentration of 0.8 μg/mL using a HRP coupled secondary antibody, as can be seen in Western blot FIG. 1. Directly HRP labeled AbD56219sco-1 also detected 10 ng of purified SNAP-25 and SNAP-25 from 3,000 cultured hIPSC MNs exposed to 3 LD₅₀ U/mL of BoNT/A with high specificity at an antibody concentration of 0.8 μg/mL, without amplification from a secondary antibody, as can be seen in Western blot FIG. 3. AbD56219sco-1 also detected SNAP-25 from 3,000 cultured hIPSC MNs at an antibody concentration of 0.4 μg/mL, using a HRP coupled secondary antibody for amplification, as can be seen in Western blot FIG. 2B. Signal was comparable to that obtained using the antibody at a concentration of 0.8 μg/mL, using the same exposure settings, as can be seen in Western blot FIG. 2A.

### II. Example 2—Cell-Based Assay Results

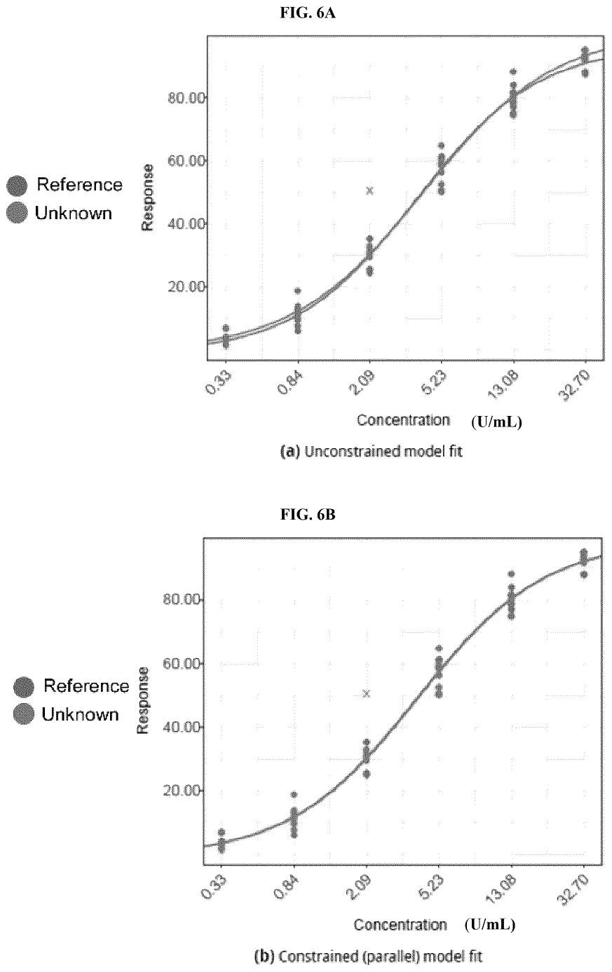

To determine the potency of a BoNT/A batch, the BoNT/A batch of unknown potency is serially diluted in the same manner as a reference BoNT/A batch of known potency. 3,000 cultured hIPSC MNs are treated with one of the dilutions of the reference or unknown BoNT/A batch, and the ratio of cleaved to un-cleaved SNAP-25 is measured using the presently disclosed anti-SNAP-25 antibody for protein detection. The “response” is the ratio of cleaved to uncleaved SNAP-25, which is plotted against the reference BoNT/A concentration, expressed in U/mL. Plotted data is fit with an unconstrained model fit (FIG. 6A), or a constrained model fit (FIG. 6B) using QuBase biostatistics software. EC₅₀ is the concentration of BoNT/A that corresponds to the midpoint on the curve. The difference in EC₅₀ values between a reference and an unknown BoNT/A batch reveals the relative strength of the unknown BoNT/A batch.

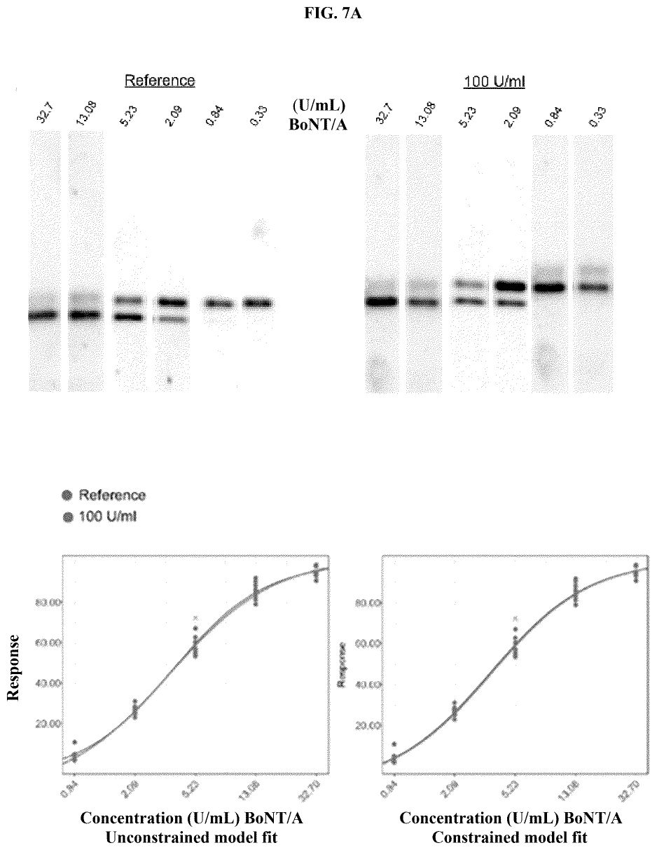

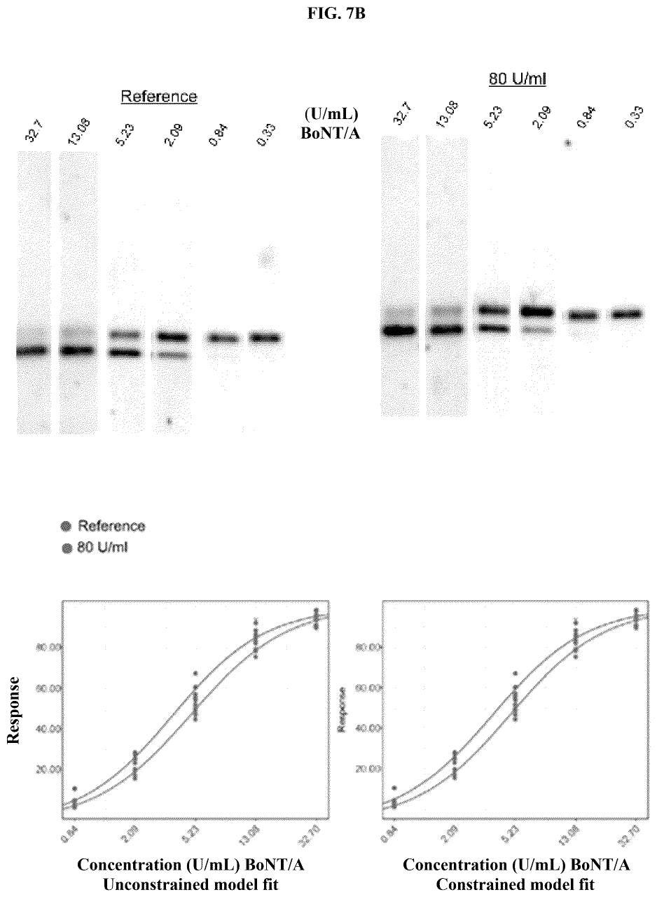

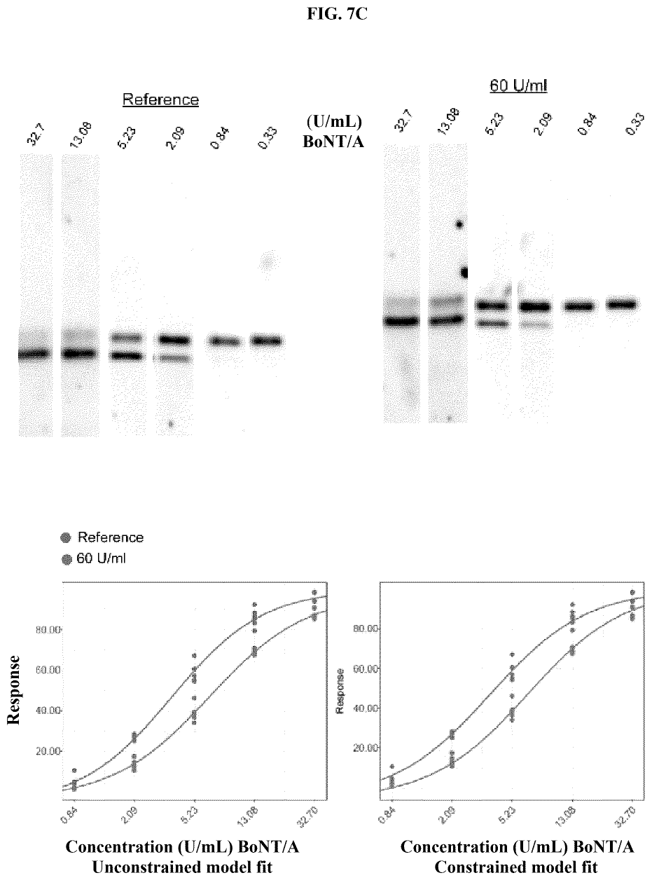

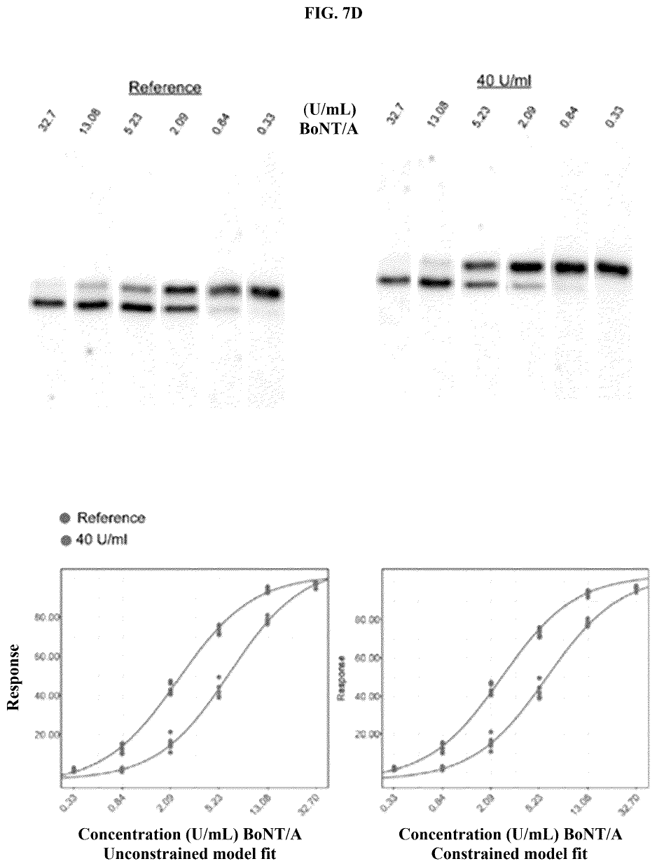

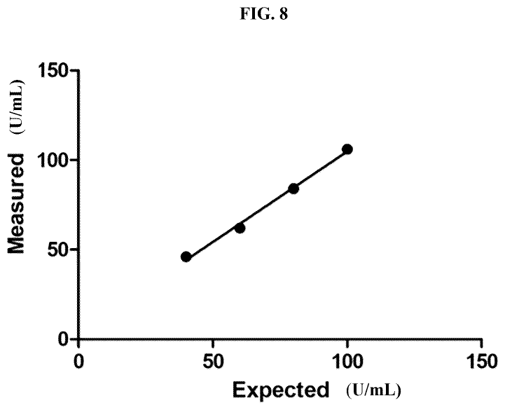

To evaluate the dynamic range of the cell-based assay using the presently disclosed antibody, the above method was repeated with a reference BoNT/A having an initial potency of 109 U/mL and four reduced potency BoNT/As having initial potencies of 100 U/mL (FIG. 7A), 80 U/mL (FIG. 7B), 60 U/mL (FIG. 7C), and 40 U/mL (FIG. 7D). The Western blots show cleaved (lower band) and uncleaved (upper band) SNAP-25, with the left Western blot lanes corresponding to reference BoNT/A concentrations of 32.70 U/mL, 13.08 U/mL, 5.23 U/mL, 2.09 U/mL, 0.84 U/mL, and 0.33 U/mL, and the right Western blot lanes corresponding to the equivalent dilution of the reduced potency BoNT/As. The response was calculated, plotted, and fit with an unconstrained model fit (left graph), and a constrained model fit (right graph). The presently disclosed antibody enabled the cell-based assay to reliably quantify the potency of samples between 40-100 U/mL, as evidenced by the linear regression (R²>0.995) of expected versus measured potencies shown in FIG. 8.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. An antibody fragment that binds to Synaptosomal-Associated Protein, 25 kDa (SNAP-25) and comprises a heavy chain region and optionally comprises a light chain region,

2. The antibody fragment of claim 1, wherein the antibody fragment is selected from a Fd, a Fv, a Fab, a Fab′, a F(ab′)₂, and a scFv.

3. The antibody fragment of claim 1, wherein the heavy chain region comprises a portion of an amino acid sequence that comprises at least 90% sequence identity to
(SEQ ID NO: 7)
EVQLLESGGGLVQPGGSLRLSCAASGFTFSSYAMHWVRQAPGKGLEWVSV
ISYDGSYTYYADSVKGRFTISRDNSKNTLYLQMNSLRAEDTAVYYCARIS
PQARGADFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKS
or
(SEQ ID NO: 8)
EVQLLESGGGLVQPGGSLRLSCAASGFTFSSYAMHWVRQAPGKGLEWVSV
ISYDGSYTYYADSVKGRFTISRDNSKNTLYLQMNSLRAEDTAVYYCARIS
PQARGADFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSEFDYKDDDDKGGSVPTIVMVDAYKRYK
GAPHHHHHH.

4. The antibody fragment of claim 1, wherein the light chain region comprises a portion of an amino acid sequence that comprises at least 90% sequence identity to
(SEQ ID NO: 9)
DIELTQPPSVSVSPGQTASITCSGDAIPSKYVSWYQQKPGQAPVLVIYRD
SDRPSGIPERFSGSNSGNTATLTISGTQAEDEADYYCGSWDMHLWVFGGG
TKLTVLGQPKAAPSVTLFPPSSEELQANKATLVCLISNFYPGAVTVAWKA
DSSPVKAGVETTTPSKQSNNKYAASSYLSLTPEQWKSHRSYSCQVTHEGS
TVEKTVAPTEA.

5. The antibody fragment of claim 1, wherein the heavy chain region comprises a portion of an amino acid sequence with 1-10 amino acid substitutions, deletions, or additions to the amino acid sequence of SEQ ID NO: 7.

6. The antibody fragment of claim 1, wherein the light chain region comprises a portion of an amino acid sequence with 1-10 amino acid substitutions, deletions, or additions to the amino acid sequence of SEQ ID NO: 9.

7. The antibody fragment of claim 1, wherein the heavy chain region comprises a portion of the amino acid sequence of SEQ ID NO: 7.

8. The antibody fragment of claim 1, wherein the light chain region comprises a portion of the amino acid sequence of SEQ ID NO: 9.

9. The antibody fragment of claim 1, wherein the antibody fragment is a fragment of a monoclonal antibody.

10. The antibody fragment of claim 1, wherein the antibody fragment is a fragment of an IgG antibody.

11. The antibody fragment of claim 1, wherein the antibody fragment is conjugated to a detectable label.

12. The antibody fragment of claim 11, wherein the detectable label comprises a radioisotope, a dye, a fluorophore, a protein, or an enzyme.

13. A nucleic acid encoding the antibody fragment of claim 1.

14. The nucleic acid of claim 13, wherein the nucleic acid comprises a region encoding the heavy chain region comprising at least 90% sequence identity to
(SEQ ID NO: 10)
GAAGTGCAATTGCTGGAAAGCGGCGGTGGCCTGGTGCAGCCGGGTGGCAG
CCTGCGTCTGAGCTGCGCGGCGTCCGGATTCACCTTTTCTTCTTACGCTA
TGCATTGGGTGCGCCAGGCCCCGGGCAAAGGTCTCGAGTGGGTTTCCGTT
ATCTCTTACGACGGTTCTTACACCTACTATGCGGATAGCGTGAAAGGCCG
CTTTACCATCAGCCGCGATAATTCGAAAAACACCCTGTATCTGCAAATGA
ACAGCCTGCGTGCGGAAGATACGGCCGTGTATTATTGCGCGCGTATCTCT
CCGCAGGCTCGTGGTGCTGACTTCGATCTGTGGGGCCAAGGCACCCTGGT
GACTGTTAGCTCAGCGTCGACCAAAGGCCCGAGCGTGTTTCCGCTGGCCC
CGAGCAGCAAAAGCACCAGCGGCGGCACCGCCGCACTGGGCTGCCTGGTG
AAAGATTATTTCCCGGAACCAGTGACCGTGAGCTGGAACAGCGGTGCCCT
GACCAGCGGCGTGCATACCTTTCCGGCGGTGCTGCAAAGCAGCGGCCTGT
ATAGCCTGAGCAGCGTTGTGACCGTGCCGAGCAGCAGCCTGGGCACCCAG
ACCTATATTTGCAACGTCAACCATAAACCGAGCAACACCAAAGTCGATAA
AAAAGTCGAACCGAAAAGCGAATTCGACTATAAAGATGACGATGACAAAG
GTGGTTCTGTTCCTACTATTGTTATGGTGGACGCCTACAAACGCTATAAG
GGCGCGCCGCACCATCATCACCATCAC.

15. The nucleic acid of claim 13, wherein the nucleic acid comprises a region encoding the light chain region comprising at least 90% sequence identity to
(SEQ ID NO: 11)
GATATCGAACTGACCCAGCCGCCGAGCGTGAGCGTGAGCCCGGGCCAGAC
CGCGAGCATTACCTGTAGCGGCGATGCTATCCCGTCTAAATACGTTTCTT
GGTACCAGCAGAAACCGGGCCAGGCGCCGGTGCTGGTGATCTACCGTGAC
TCTGACCGTCCAAGCGGCATCCCGGAACGTTTTAGCGGATCCAACAGCGG
CAACACCGCGACCCTGACCATTAGCGGCACCCAGGCGGAAGACGAAGCGG
ATTATTACTGCGGTTCTTGGGACATGCATCTGTGGGTGTTTGGCGGCGGC
ACGAAGTTAACCGTTCTTGGCCAGCCGAAAGCCGCCCCAAGCGTGACCCT
GTTTCCGCCGAGCAGCGAAGAACTGCAAGCCAACAAAGCCACCCTGGTTT
GCCTGATCAGCAATTTTTATCCGGGTGCCGTGACCGTGGCCTGGAAAGCC
GATAGCAGCCCGGTGAAAGCCGGCGTGGAAACCACCACCCCGAGCAAACA
GAGCAACAACAAATATGCCGCCAGCAGCTATCTGAGCCTGACCCCGGAAC
AGTGGAAAAGCCATCGCAGCTATAGTTGTCAAGTGACCCATGAAGGCAGC
ACCGTGGAAAAAACCGTGGCCCCGACCGAGGCC.

16. The nucleic acid of claim 13, wherein the nucleic acid comprises SEQ ID NO: 10, SEQ ID NO: 11, or a combination thereof.

17. An expression vector comprising the nucleic acid of claim 13 operably linked to a promoter.

18. A method of quantifying SNAP-25 cleavage by botulinum neurotoxins (BoNTs), the method comprising:
(a) incubating in a first series of containers a first population of cells that express SNAP-25 with a serial dilution of a first botulinum neurotoxin (BoNT) sample of known potency and incubating in a second series of containers a second population of cells that express SNAP-25 with a second serial dilution of a BoNT sample of unknown potency, wherein each incubation is for a defined period of time;
(b) after the incubation, separating cleaved SNAP-25 from uncleaved SNAP-25 from each of the first population of cells and the second population of cells;
(c) contacting cleaved and uncleaved SNAP-25 from each of the first population of cells and the second population of cells with an anti-Synaptosomal-Associated Protein, 25 kDa (SNAP-25) antibody or antigen-binding fragment thereof that comprises a heavy chain region and optionally comprises a light chain region,
and quantifying the relative amount of cleaved and uncleaved SNAP-25 in each of the first population of cells and the second population of cells.

19. The method according to claim 18, wherein a third BoNT sample, a quality control sample, of known potency, is distributed to a third container and utilized as a positive control.

20. The method of claim 18, wherein separating cleaved SNAP-25 from uncleaved SNAP-25 from each of the first population of cells and the second population of cells comprises gel electrophoresis.


SEQUENCE LISTING (15 sequences)

SEQ ID NO: 1 - protein, 5 aa, synthetic construct
SYAMH

SEQ ID NO: 2 - protein, 17 aa, synthetic construct
VISYDGSYTYYADSVKG

SEQ ID NO: 3 - protein, 12 aa, synthetic construct
ISPQARGADFDL

SEQ ID NO: 4 - protein, 11 aa, synthetic construct
SGDAIPSKYVS

SEQ ID NO: 5 - protein, 7 aa, synthetic construct
RDSDRPS

SEQ ID NO: 6 - protein, 9 aa, synthetic construct
GSWDMHLWV

SEQ ID NO: 7 - protein, 223 aa, synthetic construct
EVQLLESGGGLVQPGGSLRLSCAASGFTFSSYAMHWVRQAPGKGLEWVSVISYDGSYTYYADSVKGRFTISRDNSKNTLYLQMNSLRAEDTAVYYCARISPQARGADFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 8 - protein, 259 aa, synthetic construct
EVQLLESGGGLVQPGGSLRLSCAASGFTFSSYAMHWVRQAPGKGLEWVSVISYDGSYTYYADSVKGRFTISRDNSKNTLYLQMNSLRAEDTAVYYCARISPQARGADFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGGSVPTIVMVDAYKRYKGAPHHHHHH

SEQ ID NO: 9 - protein, 211 aa, synthetic construct
DIELTQPPSVSVSPGQTASITCSGDAIPSKYVSWYQQKPGQAPVLVIYRDSDRPSGIPERFSGSNSGNTATLTISGTQAEDEADYYCGSWDMHLWVFGGGTKLTVLGQPKAAPSVTLFPPSSEELQANKATLVCLISNFYPGAVTVAWKADSSPVKAGVETTTPSKQSNNKYAASSYLSLTPEQWKSHRSYSCQVTHEGSTVEKTVAPTEA

SEQ ID NO: 10 - DNA, 777 bp, synthetic construct
  source 1..777: mol_type = other DNA
gaagtgcaattgctggaaagcggcggtggcctggtgcagccgggtggcagcctgcgtctgagctgcgcggcgtccggattcaccttttcttcttacgctatgcattgggtgcgccaggccccgggcaaaggtctcgagtgggtttccgttatctcttacgacggttcttacacctactatgcggatagcgtgaaaggccgctttaccatcagccgcgataattcgaaaaacaccctgtatctgcaaatgaacagcctgcgtgcggaagatacggccgtgtattattgcgcgcgtatctctccgcaggctcgtggtgctgacttcgatctgtggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcgactataaagatgacgatgacaaaggtggttctgttcctactattgttatggtggacgcctacaaacgctataagggcgcgccgcaccatcatcaccatcac

SEQ ID NO: 11 - DNA, 633 bp, synthetic construct
  source 1..633: mol_type = other DNA
gatatcgaactgacccagccgccgagcgtgagcgtgagcccgggccagaccgcgagcattacctgtagcggcgatgctatcccgtctaaatacgtttcttggtaccagcagaaaccgggccaggcgccggtgctggtgatctaccgtgactctgaccgtccaagcggcatcccggaacgttttagcggatccaacagcggcaacaccgcgaccctgaccattagcggcacccaggcggaagacgaagcggattattactgcggttcttgggacatgcatctgtgggtgtttggcggcggcacgaagttaaccgttcttggccagccgaaagccgccccaagcgtgaccctgtttccgccgagcagcgaagaactgcaagccaacaaagccaccctggtttgcctgatcagcaatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

SEQ ID NO: 12 - protein, 8 aa, synthetic construct
DYKDDDDK

SEQ ID NO: 13 - protein, 14 aa, synthetic construct
VPTIVMVDAYKRYK

SEQ ID NO: 14 - protein, 6 aa, synthetic construct
HHHHHH

SEQ ID NO: 15 - protein, 206 aa, Homo sapiens
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG